(19) United States
(12) Patent Application Publication
(10) Pub. No.: US 2026/0224602 A1
(43) Pub. Date: Aug. 6, 2026

(54) METHODS AND PHARMACEUTICAL COMPOSITIONS FOR TREATING DEMENTIA

(71) Applicant: Neurxstem, Heath, OH (US)

(72) Inventor: Rene ANAND, Westerville, OH (US)

(21) Appl. No.: 19/155,158

(22) PCT Filed: Feb. 9, 2024
(86) PCT No.: PCT/US2024/015284

§ 371 Date: Aug. 8, 2025

Related U.S. Application Data
(60) Provisional application No. 63/444,531, filed on Feb. 9, 2023.
(60) Provisional application No. 63/445,248, filed on Feb. 13, 2023.

(51) Int. Cl. A61K 31/7076; A61K 31/137; A61K 31/501; A61K 31/522

(52) U.S. Cl. CPC A61K 31/7076; A61K 31/137; A61K 31/501; A61K 31/522

(57) ABSTRACT

Methods for using gene expression changes and mutations in neural organoids to assess effectiveness of treatment methodologies and identify effective pharmaceutical compositions thereof for treating dementia. Pharmaceutical compositions and methods of treatment using such pharmaceutical compositions are also provided.

### FIELD OF THE INVENTION

This disclosure provides methods for reducing or ameliorating disease severity in an individual having dementia and specific diseases thereof including Alzheimer's Disease (AD), Alzheimer's Disease Related Dementia (ADRD) or Mixed Etiology Dementia (MED). The methods provided herein include administration of therapeutically effective amounts of pharmaceutical compositions for reducing or ameliorating disease severity in an individual having dementia wherein said compositions are capable of correcting or reducing dysfunctional expression of genes related to dementia in vitro in a neural organoid platform. Further provided are pharmaceutical compositions comprising one or a plurality of active pharmaceutical ingredients alone or in combination to achieve reduction or amelioration of dementia.

### BACKGROUND OF THE INVENTION

The human brain, and diseases associated with it have been the object of investigation and study by scientists for decades. Throughout this time, neurobiologists have attempted to increase their understanding of the brain's capabilities and functions. Neuroscience has typically relied on experimental manipulation of living brains or tissue samples, but a number of factors have limited scientific progress. For ethical and practical reasons, obtaining human brain tissue is difficult while most invasive techniques are impossible to use on humans whilst they are still alive. Experiments in animals are expensive and time-consuming and many animal experiments are conducted in rodents, which have a brain structure and development that vary greatly from humans. Results obtained in animals must be verified in long and expensive human clinical trials and much of the time such animal disease models are not fully representative of disease pathology in the human brain.

Humans and other mammals are subject to a variety of forms of dementia, characterized generally as a diminution or cessation of memory and functional cognition. Examples of specific diseases characterized as dementia include Alzheimer's Disease (AD), which is a common form of irreversible degenerative brain disorder that is associated with memory loss and interferes with other intellectual abilities that complicate daily life. Alzheimer's disease accounts for 60 to 80 percent of dementia cases. Disease onset occurs most often for individuals in their mid-60s and is estimated to affect approximately five million individuals at present. However, disease onset occurs many years prior to physical expression of symptoms. The cost to society currently exceeds $270 billion and no effective treatment currently exists.

The etiology of AD is thought to involve two abnormal structures, plaques and tangles, that damage and kill nerve cells in human brain. Plaques are deposits of beta-amyloid protein fragments that build up in the spaces between nerve cells, while tangles are twisted fibers of tau, a protein that builds up inside cells. In addition, anatomical examination reveals a loss of neuronal connections in most AD patients. The result is a loss of cognitive function and the ability to perform easily normal daily activities. Thus, AD patients need extensive caregiver assistance. As a result AD is a significant financial, physical and emotional burden and one of the top causes of death in the United States.

Of the many extant neurological diseases characterized as dementia, Alzheimer's Disease, Alzheimer's Disease Related Dementia (ADRD) or Mixed Etiology Dementia (MED) have resisted successful treatment despite considerable and focused efforts (Servick, 2021, Science 372:1141). Thus, there remains a need in the art to develop therapeutically effective dementia treatments.

### SUMMARY OF THE INVENTION

This invention provides methods for reducing or ameliorating disease severity in an individual having dementia, specifically including but not limited to Alzheimer's Disease, Alzheimer's Disease Related Dementia (AD/ADRD) or Mixed Etiology Dementia (MED) comprising administering to the individual a therapeutically effective amount of a pharmaceutical composition comprising a drug capable of reducing or correcting dysfunctional expression of genes related to AD/ADRD or MED in vitro in a neural organoid platform and accordingly reduces or ameliorates disease severity in individuals having AD/ADRD or Mixed MED. Specifically, the methods provided herein rely upon identification of genes related to dementia and specifically AD/ADRD or MED that show dysfunctional or altered gene expression in neural organoid platforms in vitro for which the drugs disclosed herein are capable of reducing or reversing this dysfunctional expression. In particular embodiments these genes are set forth in Tables 1, 3, C, and 4.

A particular class of drugs provided herein are agonists of adenosine A2a receptor. (Genbank Accession No. NP_001265429.1) encoded by ADORA2A (Genbank Accession No. NM_001278497.2). In specific embodiments, such drugs include methyl (1R,4r)-4-(3-(6-amino-9-((2R,3R,4S,5S)-5-(ethylcarbamoyl)-3,4-dihydroxytetrahydrofuran-2-yl)-9H-purin-2-yl) prop-2-yn-1-yl)cyclohexane-1-carboxylate (known in the art as apadenoson). In other specific embodiments, such drugs include 2-p-(2-Carboxyethyl) phenethylamino-5′-N-ethylcarboxamidoadenosine hydrochloride hydrate (known in the art as CGS-21680). In addition to such particular and specific embodiments this disclosure encompasses related drug molecules that are agonists of adenosine A2a receptors encoded by ADORA2A and allelic variants thereof, particularly such variants associated with Alzheimer's Disease Related Dementia (AD/ADRD) or Mixed Etiology Dementia (MED).

The invention also provides pharmaceutical compositions of drugs capable of reducing or ameliorating disease severity in an individual having dementia, specifically including but not limited to Alzheimer's Disease, Alzheimer's Disease Related Dementia (AD/ADRD) or Mixed Etiology Dementia (MED). In specific embodiments the invention provides pharmaceutical compositions comprising therapeutically effective amounts of drugs capable of reducing or correcting dysfunctional expression of genes related to AD/ADRD or MED. As disclosed herein, such genes are identified from a neural organoid platform wherein administration of the drugs in vitro reduces or corrects this dysfunctional gene expression toward normalcy, and accordingly ameliorates disease severity in an individual having AD/ADRD or MED when the pharmaceutical composition is administered to such patients. In particular embodiments these genes are set forth in Tables 1, 3, C, and 4.

In specific embodiments the pharmaceutical compositions comprise drugs that are agonists of adenosine A2a receptor. (Genbank Accession No. NP_001265429.1) encoded by ADORA2A (Genbank Accession No. NM_001278497.2). In specific embodiments, such drugs include methyl (1R,4r)-4-(3-(6-amino-9-((2R,3R,4S,5S)-5-(ethylcarbamoyl)-3,4-dihydroxytetrahydrofuran-2-yl)-9H-purin-2-yl) prop-2-yn-1-yl)cyclohexane-1-carboxylate (known in the art as apadenoson). In other specific embodiments, such drugs include 2-p-(2-Carboxyethyl) phenethylamino-5′-N-ethylcarboxamidoadenosine hydrochloride hydrate (known in the art as CGS-21680). In addition to such particular and specific embodiments this disclosure encompasses related drug molecules that are agonists of adenosine A2a receptors encoded by ADORA2A and allelic variants thereof, particularly such variants associated with Alzheimer's Disease, Alzheimer's Disease Related Dementia (ADRD) or Mixed Etiology Dementia (MED).

Additional embodiments of the methods and pharmaceutical compositions disclosed herein comprise combinations of adenosine A2a receptor agonists as disclosed herein as well as combinations of these drugs with other medicaments useful for reducing or ameliorating disease severity in an individual having dementia, specifically including but not limited to Alzheimer's Disease, Alzheimer's Disease Related Dementia (ADRD) or Mixed Etiology Dementia (MED). In specific embodiments such combinations include methyl (1R,4r)-4-(3-(6-amino-9-((2R,3R,4S,5S)-5-(ethylcarbamoyl)-3,4-dihydroxytetrahydrofuran-2-yl)-9H-purin-2-yl) prop-2-yn-1-yl)cyclohexane-1-carboxylate (known in the art as apadenoson), 2-p-(2-Carboxyethyl) phenethylamino-5′-N-ethylcarboxamidoadenosine hydrochloride hydrate (known in the art as CGS-21680), in therapeutically useful combinations and dosages thereof, as well as combinations with alternative medicaments capable of reducing or ameliorating disease severity in an individual having Alzheimer's Disease Related Dementia (AD/ADRD) or Mixed Etiology Dementia (MED). In certain embodiments such alternative medicaments include Fatty Acid Amide Hydrolase (FAAH) inhibitors. In other embodiments such alternative medicaments include sphingosine-1-phosphate receptor 1 modulators.

The invention also provides pharmaceutical compositions of drugs capable of reducing or ameliorating disease severity in an individual having dementia, specifically including but not limited to Alzheimer's Disease, Alzheimer's Disease Related Dementia (AD/ADRD) or Mixed Etiology Dementia (MED), wherein a particular class of drugs provided herein are antagonists of adenosine A2b receptor (Genbank Accession No. NP_0006667.1) encoded by ADORA2B (Genbank Accession No. NM 000676.4). Specific embodiments of drugs comprising these adenosine A2b receptor antagonists include but are not limited to PBF-1129 and MRS-1706. In addition to such particular and specific embodiments this disclosure encompasses related drug molecules that are antagonists of adenosine A2b receptors encoded by ADORA2B and allelic variants thereof, particularly such variants associated with Alzheimer's Disease Related Dementia (AD/ADRD) or Mixed Etiology Dementia (MED).

The invention also provides methods for reducing or ameliorating disease severity in an individual having dementia, by treating individuals with pharmaceutical compositions of drugs capable of reducing or ameliorating disease severity in an individual having dementia, specifically including but not limited to Alzheimer's Disease, Alzheimer's Disease Related Dementia (AD/ADRD) or Mixed Etiology Dementia (MED), having a particular effect of such drugs on endocannabinoid anandamide receptor having an amino acid sequence identified by Genbank Accession No. NP_001357474.1 and is encoded by CNR1 (Genbank Accession No. NM_016083.6). Consequently, PF-3845 treatment can increase endocannabinoid anandamide receptor activity despite dementia-associated downregulation and have effects on gene expression dysregulation affected by PF-3845 treatment.

These and other data findings, features, and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE FIGURES

FIG. 1A is a diagram setting forth the rationale for producing a neural organoid platform (NNOP) as used herein shown in a micrograph of a 4× dark field image of a NOP.

FIG. 1B is a photomicrograph of a NNOP illustrated by comparison with a diagram of a 5-week-old fetus and corresponding anatomical structures.

FIG. 2 is a diagram illustrating the strategy for identifying gene expression and metabolic profiles in NNOP related to Alzheimer's Disease Related Dementia (ADRD) or Mixed Etiology Dementia (MED) for use in drug development.

FIG. 3 shows results of transcriptomic data produced using NNOP from familial amyloid plaque disorders (APP) and sporadic (SPOR) donor. Each dot is an RNA expression level of each gene. Each X and Y axis shows RPKM (Reads Per Kilo base per Million reads) which represents a gene expression level of each gene from two randomly selected out of hundreds of organoids. Independent NNOP sample transcriptomic data are shown at different time points in development. Plots represent high data reproducibility for ~15,000 genes expressed at 12 weeks in culture (W12) with a variance of <0.95 in independent replicates.

FIG. 4 shows ¹H NMR metabolomic results to track changes in metabolites of physiological significance in ADRD/MED-APP-NNOP. Independent (bottom; blue; organoid #1) (top; red; organoid #2) samples traces of ADRD/MED-APP-NNOP show high reproducibility (peak area replicability), and thus reduced to practice feasibility of tracking changes in metabolites of interest in AD/ADRD in response to any therapeutic drug.

### DETAILED DESCRIPTION

Unless defined otherwise, all technical and scientific terms used herein have the same meaning as is commonly understood by one of ordinary skill in the art. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). These references are intended to be exemplary and illustrative and not limiting as to the source of information known to the worker of ordinary skill in this art. As used herein, the following terms have the meanings ascribed to them below, unless specified otherwise.

It is noted here that as used in this specification and the appended claims, the singular forms “a,” “an,” and “the” also include plural reference, unless the context clarity dictates otherwise.

The term “about” or “approximately” means within 25%, such as within 20% (or 5% or less) of a given value or range.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention, it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “neural organoid” and “neural organoid platform” mean a non-naturally occurring three-dimensional organized cell mass that is cultured in vitro from a human induced pluripotent stem cell and develops similarly to the human nervous system in terms of neural marker expression and structure. Further a neural organoid has two or more regions. The first region expresses cortical or retinal marker or markers. The remaining regions each express markers of the brain stem, cerebellum, and/or spinal cord. Neural organoids falling within this definition herein include organoids as disclosed in U.S. Pat. No. 11,345,890, incorporated herein in its entirety.

Neural markers are any protein or polynucleotide expressed consistent with a cell lineage. By “neural marker” is meant any protein or polynucleotide, the expression of which is associated with a neural cell fate. Exemplary neural markers include markers associated with the hindbrain, midbrain, forebrain, or spinal cord. One skilled in the art will understand that neural markers are representative of the cerebrum, cerebellum and brainstem regions. Exemplary brain structures that express neural markers include the cortex, hypothalamus, thalamus, retina, medulla, pons, and lateral ventricles. Further, one skilled in the art will recognize that within the brain regions and structures, granular neurons, dopaminergic neurons, GABAergic neurons, cholinergic neurons, glutamatergic neurons, serotonergic neurons, dendrites, axons, neurons, neuronal, cilia, purkinje fibers, pyramidal cells, spindle cells, express neuronal markers. One skilled in the art will recognize that this list is not all encompassing and that neural markers are found throughout the central nervous system including other brain regions, structures, and cell types.

As set forth herein gene expression markers for human dementia, specifically including but not limited to Alzheimer's Disease, Alzheimer's Disease Related Dementia (ADRD) or Mixed Etiology Dementia (MED) are set forth in Tables 1, 3, C and 4 herein.

### Neural Organoids

Neural organoids are generated in vitro from patient tissue samples. Neural organoids were previously disclosed in U.S. Pat. No. 11,345,890, incorporated herein in its entirety. A variety of tissues can be used including skin cells, hematopoietic cells, or peripheral blood mononuclear cells (PBMCs) or in vivo stem cells directly. One of skill in the art will further recognize that other tissue samples can be used to generate neural organoids. Use of neural organoids permits study of neural development in vitro. In one embodiment skin cells are collected in a petri dish and induced to an embryonic-like pluripotent stem cell (iPSC) that have high levels of developmental plasticity. iPSCs are grown into neural organoids in said culture under appropriate conditions as set forth herein and the resulting neural organoids closely resemble developmental patterns similar to human brain. In particular, neural organoids develop anatomical features of the retina, forebrain, midbrain, hindbrain, and spinal cord. Importantly, neural organoids express >98% of the about 15,000 transcripts found in the adult human brain. iPSCs can be derived from the skin or blood cells of humans identified with dementia postmortem.

In one embodiment, the about 12-week old iPSC-derived human neural organoid has ventricles and other anatomical features characteristic of a 35-40 day old neonate. In an additional embodiment the about 12 week old neural organoid expresses beta 3-tubulin, a marker of axons as well as somato-dendritic Puncta staining for MAP2, consistent with dendrites. In yet another embodiment, at about 12 weeks the neural organoid displays laminar organization of cortical structures. Cells within the laminar structure stain positive for doublecortin (cortical neuron cytosol), Beta3 tubulin (axons) and nuclear staining. The neural organoid, by 12 weeks, also displays dopaminergic neurons and astrocytes.

Accordingly as noted, neural organoids permit study of human neural development in vitro. Further, the neural organoid offers the advantages of replicability, reliability and robustness, as shown herein using replicate neural organoids from the same source of iPSCs. In one aspect at least one cell sample reprogrammed to the induced pluripotent stem cell is a fibroblast derived from skin or blood cells from humans.

### Developmental Transcriptomics

A “transcriptome” is a collection of all RNA, including messenger RNA (mRNA), long non-coding RNAs (lncRNA), microRNAs (miRNA) and, small nucleolar RNA snoRNA), other regulatory polynucleotides, and regulatory RNA (lncRNA, miRNA) molecules expressed from the genome of an organism through transcription therefrom. Thus, transcriptomics is the study of the mRNA transcripts produced by the genome at a given time in any particular cell or tissue of the organism. Transcriptomics employs high-throughput techniques to analyze genome expression changes associated with development or disease. In certain embodiments, transcriptomic studies can be used to compare normal, healthy tissues and diseased tissue gene expression. In further embodiments, mutated genes or variants associated with disease or the environment can be identified.

Consistent with this, the aim of developmental transcriptomics is identifying genes associated with, or significant in, organismal development and disease and dysfunctions associated with development. During development, genes undergo up- and down-regulation as the organism develops. Thus, transcriptomics provides insight into cellular processes, and the biology of the organism.

Generally, in one embodiment RNA is sampled from the neural organoid described herein within at about one week, about four weeks, or about twelve weeks of development; most particularly RNA from all three time periods are samples. However, RNA from the neural organoid can be harvested at minutes, hours, days, or weeks after reprogramming. For instance, RNA can be harvested at about 10 minutes, 20 minutes, 30 minutes, 40 minutes, 50 minutes, and 60 minutes. In a further embodiment the RNA can be harvested 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In a further embodiment the RNA can be harvested at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or 1 week, 2 weeks, 3 weeks, 4 weeks, 5 weeks, 6 weeks, 7 weeks, 8 weeks, 9 weeks 10 weeks, 11 weeks, 12 weeks or more in culture. After enriching for RNA sequences, an expressed sequence tag (EST) library is generated and quantitated using the AmpliSeq™ technique from ThermoFisher. Exemplars of alternate technologies include RNASeq and chip-based hybridization methods. Transcript abundance in such experiments is compared in control neural organoids from healthy individuals vs. neural organoids generated from individuals with disease and the fold change in gene expression calculated and reported.

Furthermore, in one embodiment RNA from neural organoids for Alzheimer's disease, are converted to DNA libraries and then the representative DNA libraries are sequenced using exon-specific primers for 20,814 genes using the AmpliSeq™ technique available commercially from ThermoFisher. Reads in cpm <1 are considered background noise. All cpm data are normalized data and the reads are a direct representation of the abundance of the RNA for each gene.

Briefly, in one embodiment, the array consists of one or a plurality of genes used to predict risk of Alzheimer's disease. In an alternative embodiment, reads contain a plurality of genes that are used to treat Alzheimer's disease in a human, using patient-specific pharmacotherapy known to be associated with Alzheimer's disease. In one aspect, the gene libraries can be comprised of disease-specific gene as provided in Tables 1, 3, C, and/or 4 or a combination of genes in Table 1 with alternative disease specific genes. Exemplarily, changes in expression or mutation of disease-specific genes are detected using such sequencing, and differential gene expression detected thereby, qualitatively by detecting a pattern of gene expression or quantitatively by detecting the amount or extent of expression of one or a plurality of disease-specific genes or mutations thereof. Results of said assays using the AmpliSeq™ technique can be used to identify genes that can predict disease risk or onset and can be targets of therapeutic intervention. In further embodiments, hybridization assays can be used, including but not limited to sandwich hybridization assays, competitive hybridization assays, hybridization-ligation assays, dual ligation hybridization assays, or nuclease assays.

### Neural Organoids and Pharmaceutical Testing

Neural organoids are useful for pharmaceutical testing. Currently, drug screening studies including toxicity, safety and or pharmaceutical efficacy, are performed using a combination of in vitro work, rodent/primate studies and computer modeling. Collectively, these studies seek to model human responses, in particular physiological responses of the central nervous system.

Human neural organoids are advantageous over current pharmaceutical testing methods for several reasons. First neural organoids are easily derived from healthy and diseased patients, mitigating the need to conduct expensive clinical trials. Second, rodent models of human disease are unable to mimic physiological nuances unique to human growth and development. Third, use of primates creates ethical concerns. Finally, current methods are indirect indices of drug safety. Alternatively, neural organoids offer an inexpensive, easily accessible model of human brain development. This model permits direct, and thus more thorough, understanding of the safety, efficacy, and toxicity of pharmaceutical compounds.

Starting material for neural organoids is easily obtained from healthy and diseased patients. Further, because human organoids are easily grown they can be produced en mass. This permits efficient screening of pharmaceutical compounds.

Neural organoids are advantageous for identifying biomarkers of a disease or a condition, the method comprising a) obtaining a biological sample from a human patient; and b) detecting whether at least one biomarker is present in the biological sample by contacting the biological sample with an array comprising binding molecules specific for the biomarkers and detecting binding between the at least one biomarker and the specific binding molecules. In further embodiments, the biomarker serves as a gene therapy target.

### Relevance for Adenosine Receptors in Dementia Etiologies

Certain studies in the prior art suggest the possibility of a role for adenosine receptors in the progression of the neuropathological changes that are observed in AD (Kinney et al., 2018, Alzheimer's & Dementia: Translational Research & Clinical Interventions, Alzheimer's and Dementia: Translational Research and Clinical Intervention 4:575-590). For example, an association between a polymorphism of the ADORA2A gene with hippocampal volume in mild cognitive impairment and AD has been reported (Horgusluoglu-Moloch et al., 2017, Targeted neurogenesis pathway-based gene analysis identifies ADORA2A associated with hippocampal volume in mild cognitive impairment and Alzheimer's disease, Neurobiol. Aging 60:92-103). Adenosine receptors have different functions, and the A_{2A} receptor has a broader anti-inflammatory effects throughout the body, additionally (Hasko and Pather, 2008, A_{2A} receptors in inflammation and injury: lessons learned from transgenic animals, J. Leucocyte Biol. 83:447-455). Both receptors also regulate the release of dopamine and glutamate in the brain (Sun and Hwang, 2016, Adenosine A2B Receptor: From Cell Biology to Human Diseases, Front. Chem. 24:37; Fuxe et al., 2007, Adenosine receptor-dopamine receptor interactions in the basal ganglia and their relevance for brain function, Physiol Behav 92:210-7; Schiffmann et al., 2007, Adenosine A2A receptors and basal ganglia physiology, Prog Neurobiol 83:277-92; and Cunha et al., 2008, How does adenosine control neuronal dysfunction and neurodegeneration?, J. Neurobiol. 139:1019-1055).

The Adenosine A_{2A} Receptor (ADORA2A) gene is expressed at a lower level compared to normal (p<0.05; Table C) in gene expression analyses using NNOP and is consistent with this report. Furthermore, multiple genes in the STRING analysis (ADORA1; CALM3; SYNGR1 and ACTN4) show epigenetic co-regulation with the ADORA2A gene (Table C). These data strongly suggest that A_{2A} agonists could augment ADORA2A anti-inflammatory pathway function. Multiple agonists have been characterized (CGS21680; DPMA; HE-NECA; ATL-146e; and CVT-3146). As set forth herein this invention utilizes two of them, CGS-21680 and Apadenoson (ATL-146e), as therapeutic compositions for treating ADRD/MED.

This invention provides methods for reducing or ameliorating disease severity in an individual having dementia, specifically including but not limited to Alzheimer's Disease, Alzheimer's Disease Related Dementia (ADRD) or Mixed Etiology Dementia (MED) comprising administering to the individual a therapeutically effective amount of a pharmaceutical composition comprising a drug capable of reducing or correcting dysfunctional expression of genes related to ADRD or MED in vitro in a neural organoid platform and accordingly reduces or ameliorates disease severity in individuals having ADRD or Mixed MED. Specifically, the methods provided herein rely upon identification of genes related to ADRD or MED that show dysfunctional or altered gene expression in neural organoid platforms in vitro for which the drugs disclosed herein are capable of reducing this dysfunctional expression. In particular embodiments these genes are set forth in Tables 1, 3, C, and 4.

As will be understood by the skilled worker, the majority of dementia cases in the elderly (65 years of age and older) are so-called “mixed” dementias, mainly Alzheimer's disease concomitant with cerebrovascular disease and/or Lewy body formation (see, James et al., 2012, Dementia From Alzheimer Disease and Mixed Pathologies in the Oldest Old, JAMA 307:1798-1800; Schneider et al., 2007, Neurology 69:2197-2204).

As used herein, the term “dementia” is intended to encompass illness particularly in humans having symptoms including diminution or cessation of memory and functional cognition. Examples of specific diseases characterized as dementia include Alzheimer's Disease (AD), which is a common form of irreversible degenerative brain disorder that is associated with memory loss and interferes with other intellectual abilities that complicate daily life. Also included in the meaning of the term dementia as used herein are Alzheimer's Disease Related Dementia (ADRD), Mixed Etiology Dementia (MED), Lewy Body dementia, fronto-temporal dementia, or vascular contributors to dementia.

As used herein, the terms “reducing or ameliorating” are intended to be understood to include improvements or reduction in impairments or progression thereof of symptoms, particularly cognitive symptoms, of dementia, specifically including but not limited to Alzheimer's Disease, Alzheimer's Disease Related Dementia (ADRD) or Mixed Etiology Dementia (MED).

As used herein, the term “therapeutically effective amount” of a drug for treating dementia, specifically including but not limited to Alzheimer's Disease, Alzheimer's Disease Related Dementia (ADRD) or Mixed Etiology Dementia (MED) will be understood to include dosage amounts that provide improvements or reduction in impairments or progression thereof of symptoms, particularly cognitive symptoms, of dementia, specifically including but not limited to Alzheimer's Disease, Alzheimer's Disease Related Dementia (ADRD) or Mixed Etiology Dementia (MED).

As used herein, the term “dysfunctional gene expression” will be understood to mean differences in gene expression in NNOP as shown herein produced from skin cells, inter alia by methods for producing induced pluripotent stem cells that are then differentiated into NNOP s set forth in U.S. Pat. No. 11,345,890, incorporated herein in its entirety, between individuals having dementia, specifically including but not limited to Alzheimer's Disease, Alzheimer's Disease Related Dementia (ADRD) or Mixed Etiology Dementia (MED) or a genetic propensity for developing Alzheimer's Disease (AD) or Alzheimer's Disease Related Dementia (ADRD) or Mixed Etiology Dementia (MED) and individuals without Alzheimer's Disease Related Dementia (ADRD) or Mixed Etiology Dementia (MED) or a genetic propensity for developing Alzheimer's Disease Related Dementia (ADRD) or Mixed Etiology Dementia (MED). Genetically, AD is divided into familial cases and sporadic cases which can be found in patients clinically. The familial form is due to mutations in three major genes: amyloid precursor protein (APP) gene, apolipoprotein E (ApoE), or presenilin 2 gene (PSEN2). Useful NNOPs as set forth herein can be generated as disclosed herein from adult skin cells of individuals having genetic traits including those of sporadic Alzheimer's disease (identified as SPOR herein), amyloid plaque disorders (APP), mutations in ApoE (ApoE), or presenilin (PSEN2).

As used herein, the term “reducing or correcting dysfunctional gene expression” refers to changes in expression of certain genes (set forth herein in Tables 1, 3, C, and 4) in NNOP in response to addition of therapeutically effective amounts of a drug a disclosed herein, wherein dysfunctional gene expression has the meaning set forth above, and degree of effect on the NNOP will be understood to reduce the extent of the dysfunction in expression of said genes.

In particular embodiments, the genes having differential dysfunctional gene expression related to or associated with Alzheimer's Disease Related Dementia (ADRD) or Mixed Etiology Dementia (MED) are one or more of the genes set forth herein in Table 1, 3, C, and 4.

A particular class of drugs provided herein are agonists of adenosine A2a receptor. (GENBANK ACCESSION NO. NP_001265429.1) encoded by ADORA2A (GENBANK ACCESSION NO. NM_001278497.2). In specific embodiments, such drugs include methyl (1R,4r)-4-(3-(6-amino-9-((2R,3R,4S,5S)-5-(ethylcarbamoyl)-3,4-dihydroxytetrahydrofuran-2-yl)-9H-purin-2-yl) prop-2-yn-1-yl)cyclohexane-1-carboxylate (known in the art as apadenoson). In other specific embodiments, such drugs include 2-p-(2-Carboxyethyl) phenethylamino-5′-N-ethylcarboxamidoadenosine hydrochloride hydrate (known in the art as CGS-21680). In addition to such particular and specific embodiments this disclosure encompasses related drug molecules that are agonists of adenosine A2a receptors encoded by ADORA2A and allelic variants thereof, particularly such variants associated with Alzheimer's Disease Related Dementia (ADRD) or Mixed Etiology Dementia (MED).

As used herein the adenosine A2a receptor agonists include related molecules, including variants in substituents, sidechains, and the like, that retain the capacity to reduce or ameliorate disease severity in patients having dementia, specifically including but not limited to Alzheimer's Disease, Alzheimer's Disease Related Dementia (ADRD) or Mixed Etiology Dementia (MED). Such drugs also include structurally related drugs having improved specificity for any of the adenosine A2a receptors disclosed herein or known in the art to be associated with Alzheimer's Disease Related Dementia (ADRD) or Mixed Etiology Dementia (MED). Such alternative embodiments of adenosine A2a receptors associated with Alzheimer's Disease Related Dementia (ADRD) or Mixed Etiology Dementia (MED) include but are not limited to allelic variants or genetic variants found to be associated with associated with Alzheimer's Disease Related Dementia (ADRD) or Mixed Etiology Dementia (MED)

The invention also provides pharmaceutical compositions of drugs capable of reducing or ameliorating disease severity in an individual having Alzheimer's Disease Related Dementia (ADRD) or Mixed Etiology Dementia (MED). In specific embodiments the invention provides pharmaceutical compositions comprising therapeutically effective amounts of drugs capable of reducing or correcting dysfunctional expression of genes related to ADRD or MED. As disclosed herein, such genes are identified from a neural organoid platform wherein administration of the drugs in vitro reduces or alters this dysfunctional gene expression, and accordingly ameliorates disease severity in an individual having ADRD or Mixed MED when the pharmaceutical composition is administered to such patients. In particular embodiments these genes are set forth in Tables 1, 3, C, and 4 herein.

As used herein, the term “pharmaceutical composition” is intended to encompass and will be understood by those skilled in these arts to include agonists of adenosine A2a receptor, specifically adenosine A2a receptor having an amino acid sequence identified by GENBANK ACCESSION NO. NP_001265429.1 and encoded by ADORA2A (GENBANK ACCESSION NO. NM_001278497.2). Pharmaceutical compositions according to this invention include pharmacological salts, hydrates, or conjugates thereof. These compositions also include formulations, particularly formulations capable of traversing the blood-brain barrier and formulations that can be provided or oral administration for example in pill form. Alternative formulations, for example for injection, administration by inhalation, or rectal suppositories are also envisioned.

Specific embodiments of drugs comprising the active pharmaceutical ingredient (API) of the pharmaceutical compositions provided herein include methyl (1R,4r)-4-(3-(6-amino-9-((2R,3R,4S,5S)-5-(ethylcarbamoyl)-3,4-dihydroxytetrahydrofuran-2-yl)-9H-purin-2-yl) prop-2-yn-1-yl)cyclohexane-1-carboxylate (known in the art as apadenoson). In other specific embodiments, such drugs include 2-p-(2-Carboxyethyl) phenethylamino-5′-N-ethylcarboxamidoadenosine hydrochloride hydrate (known in the art as CGS-21680). In addition to such particular and specific embodiments this disclosure encompasses related drug molecules that are agonists of adenosine A2a receptors encoded by ADORA2A and allelic variants thereof, particularly such variants associated with Alzheimer's Disease Related Dementia (ADRD) or Mixed Etiology Dementia (MED).

Additional embodiments of the methods and pharmaceutical compositions disclosed herein comprise combinations of adenosine A2a receptor agonists as disclosed herein as well as combinations of these drugs with other medicaments useful for reducing or ameliorating disease severity in an individual having Alzheimer's Disease Related Dementia (ADRD) or Mixed Etiology Dementia (MED). In specific embodiments such combinations include methyl (1R,4r)-4-(3-(6-amino-9-((2R,3R,4S,5S)-5-(ethylcarbamoyl)-3,4-dihydroxytetrahydrofuran-2-yl)-9H-purin-2-yl) prop-2-yn-1-yl)cyclohexane-1-carboxylate (known in the art as apadenoson), and/or 2-p-(2-Carboxyethyl) phenethylamino-5′-N-ethylcarboxamidoadenosine hydrochloride hydrate (known in the art as CGS-21680), in therapeutically useful combinations and dosages thereof, as well as combinations with alternative medicaments capable of reducing or ameliorating disease severity in an individual having Alzheimer's Disease Related Dementia (ADRD) or Mixed Etiology Dementia (MED).

As used herein, “combinations” can include formulations comprising one or more of the drugs specifically identified herein for reducing or ameliorating disease severity in an individual having dementia, specifically including but not limited to Alzheimer's Disease, Alzheimer's Disease Related Dementia (ADRD) or Mixed Etiology Dementia (MED). In certain combinations the drugs are administered concomitantly and in other administration can be achieved over a specified time course. In some embodiments the combinations are provided in a single formulation whereas in others the drugs are separately formulated.

In certain embodiments the alternative medicament administered in combination with adenosine A2a receptor agonists as disclosed herein are Fatty Acid Amide Hydrolase (FAAH) inhibitors, in specific embodiments wherein the FAAH inhibitor is PF-3845, having the structure

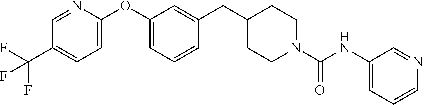 [SMILES: O=C(Nc1cccnc1)N1CCC(Cc2cccc(Oc3ccc(C(F)(F)F)cn3)c2)CC1]

SSR411298 (Clinical trials for SSR411298, EU Clinical Trials Register), or PF-622 (Ahn et al., 2007, Biochem. 46:13019-13030). In certain alternative embodiments the alternative medicament administered in combination with adenosine A2a receptor agonists as disclosed herein are sphingosine-1-phosphate receptor 1 modulators, wherein specific sphingosine-1-phosphate receptor 1 modulators useful in these alternative medicaments are fingolimod, siponimed (Mayzant), ponesimed (ACT 12800), ceralifimod (ONO-4641), or Amiselmod (MT-1303).

As disclosed herein, genes have been identified from a neural organoid platform wherein administration of FAAH inhibitors in combination with adenosine A2a receptor agonists as disclosed herein in vitro reduces or alters this dysfunctional gene expression, and accordingly ameliorates disease severity in an individual having ADRD or Mixed MED when the pharmaceutical composition is administered to such patients. Additionally, genes have been identified from a neural organoid platform wherein administration of 1-phosphate receptor 1 (S1PR1) modulators, wherein specific sphingosine in combination with adenosine A2a receptor agonists as disclosed herein in vitro reduces or alters this dysfunctional gene expression, and accordingly ameliorates disease severity in an individual having ADRD or Mixed MED when the pharmaceutical composition is administered to such patients. In particular embodiments these genes are set forth in Tables 3 and 4 herein.

In further embodiments useful in the methods and provided in pharmaceutical compositions provided herein are adenosine A2b receptor antagonists, wherein the adenosine A2b receptor is encoded by ADORA2B (GenBank Accession No. NM_000676.4). Specific embodiments of drugs comprising these adenosine A2b receptor antagonists include but are not limited to PBF-1129 and MRS-1706; see, Vazquez et al., 2008, “Local stimulation of the adenosine A2B receptors induces an increased release of IL-6 in mouse striatum: an in vivo microdialysis study,” J. Neurochem. 105:904-9 and Ryzhov et I., 2008, “Effect of A2B adenosine receptor gene ablation on proinflammatory adenosine signaling in mast cells,” J. Immunol. 180:7212-20.

As disclosed herein, genes have been identified from a neural organoid platform wherein administration of adenosine A2b receptor antagonists in vitro reduces or alters this dysfunctional gene expression, and accordingly ameliorates disease severity in an individual having ADRD or Mixed MED when the pharmaceutical composition is administered to such patients. In particular embodiments these genes are set forth in Tables 1, 3, C, and 4 herein.

In further embodiments the skilled worker will understand that treatment with FAAH inhibitor PF-3845 will raise endogenous levels of endocannabinoid anandamide receptor agonist anandamide, wherein the endocannabinoid anandamide receptor has an amino acid sequence identified by GenBank Accession No. NP_001357474.1 and is encoded by CNR1 (GenBank Accession No. NM_016083.6). Consequently, PF-3845 treatment can increase endocannabinoid anandamide receptor activity despite dementia-associated downregulation and can have effects on gene expression that were affected by AD. In particular embodiments these genes are set forth in Table 3 herein.

These and other data findings, features, and advantages of the present disclosure will be more fully understood from the following detailed description taken together with the accompanying claims. It is noted that the scope of the claims is defined by the recitations therein and not by the specific discussion of features and advantages set forth in the present description.

### EXAMPLES

The Examples that follow are illustrative of specific embodiments of the invention, and the use thereof. It is set forth for explanatory purposes only and is not taken as limiting the invention. In particular, the example demonstrates the effectiveness of neural organoids in predicting future disease risk.

### Materials and Methods

The neural organoids described above were developed using the following materials and methods.

### Summary of Methods

Neural Organoids derived from induced pluripotent stem cells derived from adult skin cells of AD, ADRD or MED patients were grown in vitro for 4 weeks as previous described in U.S. Pat. No. 11,435,890, incorporated by reference in its entirety herein. Transcriptomic data from these neural organoids were obtained. Differences in expression of 20,814 genes expressed in the human genome were determined between these neural organoids and those from neural organoids from a normal individual human (an individual not having/diagnosed with AD, ADRD, or MED). Detailed data analysis using Gene Card and Pubmed data bases were performed. Genes that were expressed at greater than 1.4-fold were found to be highly significant because a vast majority were correlated with genes previously associated with a multitude of neurodevelopmental and neurodegenerative diseases as well as those found to be dysregulated in postmortem patient brains. These genes comprise a suite of biomarkers for Alzheimer's disease.

Cells used in these methods include human iPSCs, feeder-dependent (System Bioscience. WT SC600A-W) and CF-1 mouse embryonic fibroblast feeder cells, gamma-irradiated (Applied StemCell, Inc #ASF-1217)

Growth media, or DMEM media, used in the examples contained the supplements as provided in Table A (Growth Media and Supplements used in Examples).

TABLE A
Growth Media and Supplements used in Examples

| Media/Supplement | Vendor/Catalog Number |
|---|---|
| DMEM non-essential amino acids | MEM-NEAA, Invitrogen |
| | #11140-050 |
| Phosphate Buffered Saline, sterile | Invitrogen #14040-091 |
| Phosphate Buffered Saline, Ca++ | Invitrogen #14190-094 |
| and Mg++ free | |
| Gentamicin Reagent Solution | Invitrogen #15750-060 |
| Antibiotic-Antimycotic | Invitrogen #15240-062 |
| 2-mercaptoethanol | EmbryoMAX, |
| | EMBMillipore#ES-007-E |
| Basic fibroblast growth factor | FGF, PeproTech #051408-1 |
| Heparin | Sigma, #H3149-25KU |
| Insulin solution | Sigma #19278-5 ml |
| Dimethyl sulfoxide | Millipore #D9170-5VL |
| ROCK Inhibitor Y27632 | Millipore#SCM075 |
| Gelatin solution, Type B | Sigma #GI 393-100 ml |
| Matrigel Matrix NOT Growth | BD Bioscience #354234 |
| Factor Reduced Matrigel | |
| Accutase | Sigma #A6964 |
| Hydrogen Peroxide | Fisher #H325-500 |
| Ethanol | |
| Sterile H20 | |

One skilled in the art will recognize that additional formulations of media and supplements can be used to culture, induce and maintain pluripotent stem cells and neural organoids.

Experimental protocols required the use of multiple media compositions including MEF Media, IPSC Media, EB Media, Neural Induction Media, and Differentiation Medias 1, 2, and 3.

Mouse embryonic fibroblast (MEF) was used in cell culture experiments. MEF Media comprised DMEM media supplemented with 10% Feta Bovine Serum, 100 units/ml penicillin, 100 microgram/ml streptomycin, and 0.25 microgram/ml Fungizone.

Induction media for pluripotent stem cells (IPSC Media) comprised DMEM/F12 media supplemented with 20% Knockout Replacement Serum, 3% Fetal Bovine Serum with 2 mM Glutamax, IX Minimal Essential Medium Nonessential Amino Acids, and 20 nanogram/ml basic Fibroblast Growth Factor

Embryoid Body (EB) Media comprised Dulbecco's Modified Eagle's Medium (DMEM) (DMEM)/Ham's F-12 media, supplemented with 20% Knockout Replacement Serum, 3% Fetal Bovine Serum containing 2 mM Glutamax, IX Minimal Essential Medium containing Nonessential Amino Acids, 55 microM beta-mercaptoethanol, and 4 ng/ml basic Fibroblast Growth Factor.

Neural Induction Media contained DMEM/F12 media supplemented with a 1:50 dilution N2 Supplement, a 1:50 dilution GlutaMax, a 1:50 dilution MEM-NEAA, and 10 microgram/ml Heparin.

Three differentiation media were used to produce and grow neural organoids. Differentiation Media 1 contained DMEM/F12 media and Neurobasal media in a 1:1 dilution. Each media is commercially available from Invitrogen. The base media was supplemented with a 1:200 dilution N2 supplement, a 1:100 dilution B27-vitamin A, 2.5 microgram/ml insulin, 55 microM beta-mercaptoethanol kept under nitrogen mask and frozen at −20° C., 100 units/ml penicillin, 100 microgram/ml streptomycin, and 0.25 microgram/ml Fungizone.

Differentiation Media 2 contained DMEM/F12 media and Neurobasal media in a 1:1 dilution supplemented with a 1:200 dilution N2 supplement, a 1:100 dilution B27 containing vitamin A, 2.5 microgram/ml Insulin, 55 umicroMolar beta-mercaptoethanol kept under nitrogen mask and frozen at −20° C., 100 units/ml penicillin, 100 microgram/ml streptomycin, and 0.25 microgram/ml Fungizone.

Differentiation Media 3 consisted of DMEM/F12 media: Neurobasal media in a 1:1 dilution supplemented with 1:200 dilution N2 supplement, a 1:100 dilution B27 containing vitamin A), 2.5 microgram/ml insulin, 55 microMolar beta-mercaptoethanol kept under nitrogen mask and frozen at −20° C., 100 units/ml penicillin, 100 microgram/ml streptomycin, 0.25 microgram/ml Fungizone, TSH, and Melatonin.

The equipment used in obtaining, culturing and inducing differentiation of pluripotent stem cells is provided in Table B (Equipment used in Experimental Procedures). One skilled in the art would recognize that the list is not at all exhaustive but merely exemplary.

TABLE B
Equipment used in Experimental Procedures.

| | |
|---|---|
| StemPro EZPassage | Invitrogen#23181-010 |
| Tissue Culture Flasks, 115 cm² reclosable | TPP #TP90652 |
| Tissue Culture Flask, 150 cm² reclosable | TPP#TP90552 |
| Lipidure coat plate, 96 wells, U-bottom | LCU96 |
| Lipidure coat MULTI dish, 24 well | 510101619 |
| Parafilm | Sigma #P7793 |
| Sterile Filtration Units for 150 ml/250 ml | Sigma #TPP99150/ |
| solutions | TPP99250 |
| Benchtop Tissue Culture Centrifuge | ThermoFisher |
| C0₂ incubator, maintained at 37° C. and 5% C0₂ | ThermoFisher |
| Bench top rotary shaker | ThermoFisher |
| Light Microscope | Nikon |
| Confocal Microscope | Nikon |

### Example 1: Generation of Human Induced Pluripotent Stem Cell-Derived Neural Organoids

Human induced pluripotent stem cell-derived neural organoids were generated according to the following protocol, as set forth in U.S. Pat. No. 11,435,890, incorporated by reference in its entirety herein. Briefly, irradiated murine embryonic fibroblasts (MEF) were plated on a gelatin coated substrate in MEF media (Dulbecco's Modified Eagle Medium (DMEM) supplemented with 10% Fetal Bovine Serum, 100 units/ml penicillin, 100 microgram/ml streptomycin, and 0.25 microgram/ml Fungizone) at a density of 2×10⁵ cells per well. The seeded plate was incubated at 37° C. overnight.

After incubation, the MEFs were washed with pre-warmed sterile phosphate buffered saline (PBS). The MEF media was replaced with 1 mL per well of induced pluripotent stem cell (iPSC) media containing Rho-associated protein kinase (ROCK) inhibitor. A culture plate with iPSCs was incubated at 37° C. The iPSCs were fed every other day with fresh iPSC media containing ROCK inhibitor. The iPSC colonies were lifted, divided, and transferred to the culture wells containing the MEF cultures so that the iPSC and MEF cells were present therein at a 1:1 ratio. Embryoid bodies (EB) were then prepared. Briefly, a 100 mm culture dish was coated with 0.1% gelatin and the dish placed in a 37° C. incubator for 20 minutes, after which the gelatin-coated dish was allowed to air dry in a biological safety cabinet. The wells containing iPSCs and MEFs were washed with pre-warmed PBS lacking Ca²⁺/Mg²⁺. A pre-warmed cell detachment solution of proteolytic and collagenolytic enzymes (1 mL/well) was added to the iPSC/MEF cells. The culture dishes were incubated at 37° C. for 20 minutes until cells detached. Following detachment, pre-warmed iPSC media was added to each well and gentle agitation used to break up visible colonies. Cells and media were collected and additional pre-warmed media added, bringing the total volume to 15 mL. Cells were placed on a gelatin-coated culture plate at 37° C. and incubated for 60 minutes, thereby allowing MEFs to adhere to the coated surface. The iPSCs present in the cell suspension were then counted.

The suspension was then centrifuged at 300×g for 5 minutes at room temperature, the supernatant discarded, and cells re-suspended in EB media supplemented with ROCK inhibitor (50 μM final concentration) and 4 ng/ml basic Fibroblast Growth Factor to a volume of 9,000 cells/150 μL. EB media is a mixture of DMEM/Ham's F-12 media supplemented with 20% Knockout Replacement Serum, 3% Fetal Bovine Serum (2 mM Glutamax), 1× Minimal Essential Medium Nonessential Amino Acids, and 55 μM beta-mercaptoethanol. The suspended cells were plated (150 μL) in a LIPIDURE® low-attachment U-bottom 96-well plate and incubated at 37° C.

The plated cells were fed every other day during formation of the embryoid bodies by gently replacing three fourths of the embryoid body media without disturbing the embryoid bodies forming at the bottom of the well. Special care was taken in handling the embryoid bodies so as not to perturb the interactions among the iPSC cells within the EB through shear stress during pipetting. For the first four days of culture, the EB media was supplemented with 50 μM ROCK inhibitor and 4 ng/ml beta-fibroblast growth factor (bFGF). During the remaining two to three days the embryoid bodies were cultured, no ROCK inhibitor or bFGF was added.

On the sixth or seventh day of culture, the embryoid bodies were removed from the LIPIDURE® 96 well plate and transferred to two 24-well plates containing 500 μL/well Neural Induction media, DMEM/F12 media supplemented with a 1:50 dilution N2 Supplement, a 1:50 dilution GlutaMax, a 1:50 dilution MEM-Non-Essential Amino Acids (NEAA), and 10 μg/ml Heparin. Two embryoid bodies were plated in each well and incubated at 37° C. The media was changed after two days of incubation. Embryoid bodies with a “halo” around their perimeter indicate neuroectodermal differentiation. Only embryoid bodies having a “halo” were selected for embedding in Matrigel, remaining embryoid bodies were discarded.

Plastic paraffin film (PARAFILM) rectangles (having dimensions of 5 cm×7 cm) were sterilized with 3% hydrogen peroxide to create a series of dimples in the rectangles. This dimpling was achieved, in one method, by centering the rectangles onto an empty sterile 200 μL tip box press, and pressing the rectangles gently to dimple it with the impression of the holes in the box. The boxes were sprayed with ethanol and left to dry in the biological safety cabinet.

Frozen Matrigel matrix aliquots (500 μL) were thawed on ice until equilibrated at 4° C. A single embryoid body was transferred to each dimple of the film. A single 7 cm×5 cm rectangle holds approximately twenty (20) embryoid bodies. Twenty microliter (20 μL) aliquots of Matrigel were transferred onto the embryoid bodies after removing extra media from the embryoid body with a pipette. The Matrigel was incubated at 37° C. for 30 min until the Matrigel polymerized. The 20 μL droplet of viscous Matrigel was found to form an optimal three-dimensional environment that supported the proper growth of the neural organoid from embryoid bodies by sequestering the gradients of morphogens and growth factors secreted by cells within the embryoid bodies during early developmental process. However, the Matrigel environment permitted exchange of essential nutrients and gases. Gentle oscillation by hand twice a day for a few minutes within a tissue culture incubator (37° C./5% CO₂) further allowed optimal exchange of gases and nutrients to the embedded embryoid bodies.

Differentiation Media 1, a one-to-one mixture of DMEM/F12 and Neurobasal media supplemented with a 1:200 dilution N2 supplement, a 1:100 dilution B27-vitamin A, 2.5 μg/mL insulin, 55 UM beta-mercaptoethanol kept under nitrogen mask and frozen at −20° C., 100 units/mL penicillin, 100 μg/mL streptomycin, and 0.25 μg/mL Fungizone, was added to a 100 mm tissue culture dish. The film containing the embryoid bodies in Matrigel was inverted onto the 100 mm dish with differentiation media 1 and incubated at 37° C. for 16 hours. After incubation, the embryoid body/Matrigel droplets were transferred from the film to the culture dishes containing media. Static culture at 37° C. was continued for 4 days until stable neural organoids formed.

Organoids were gently transferred to culture flasks containing differentiation media 2, a one-to-one mixture of DMEM/F12 and Neurobasal media supplemented with a 1:200 dilution N2 supplement, a 1:100 dilution B27+vitamin A, 2.5 μg/mL insulin, 55 microM beta-mercaptoethanol kept under nitrogen mask and frozen at −20° C., 100 units/mL penicillin, 100 μg/mL streptomycin, and 0.25 μg/mL Fungizone. The flasks were placed on an orbital shaker rotating at 40 rpm within the 37° C./5% CO₂ incubator.

The media was changed in the flasks every 3-4 days to provide sufficient time for morphogen and growth factor gradients to act on targets within the recipient cells forming relevant structures of the brains. Great care was taken when changing media so as to avoid unnecessary perturbations to the morphogen/secreted growth factor gradients developed in the outer most periphery of the organoids as the structures grew into larger organoids.

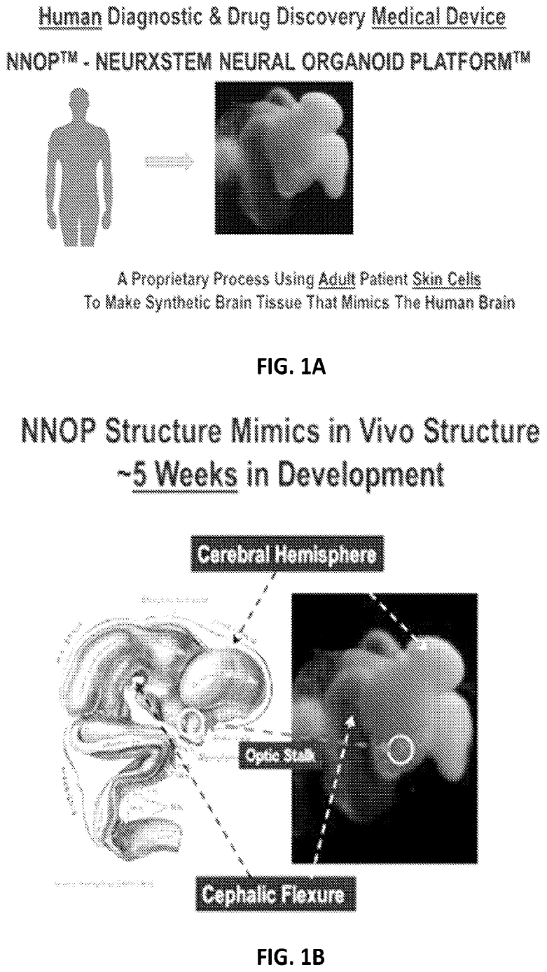

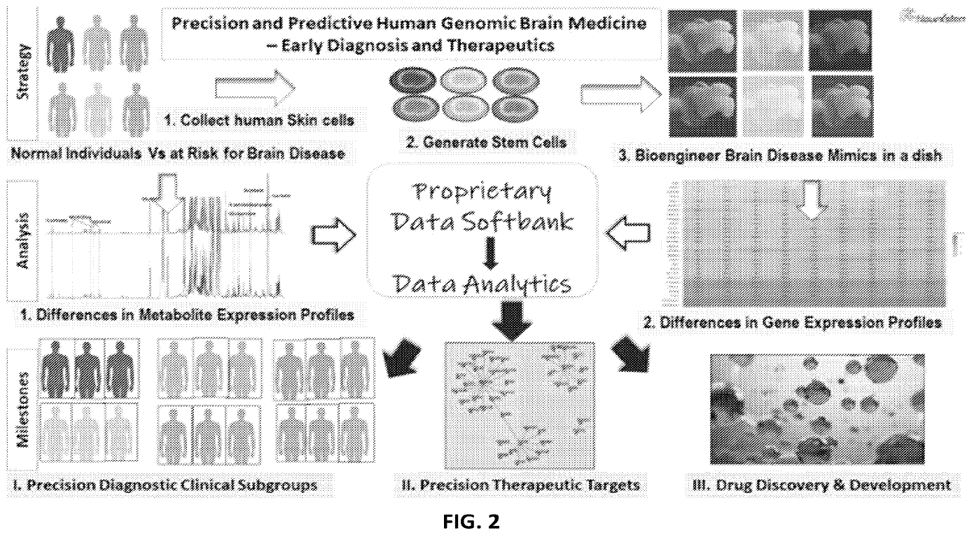

FIG. 1 illustrates neural organoid development in vitro. Based on transcriptomic analysis, iPSC cells form a body of cells after 3D culture, which become neural progenitor cells (NPC) after neural differentiation media treatment. Neurons were observed in the cell culture after about one week. After about four (4) weeks or before, neurons of multiple lineage appeared. At about twelve (12) weeks or before, the organoid developed to a stage having different types of cells, including microglia, oligodendrocyte, astrocyte, neural precursor, neurons, and interneurons.

### Example 2: Human Induced Pluripotent Stem Cell-Derived Neural Organoids Express Characteristics of Human Brain Development

After approximately 12 weeks of in vitro culture, transcriptomic and immunohistochemical analysis indicated that organoids were generated according to the methods delineated in Example 1. Specifically, the organoids contained cells expressing markers characteristic of neurons, astrocytes, oligodendrocytes, microglia, and vasculature (FIGS. 1A and 1B) and all major brain structures of neuroectodermal derivation. Morphologically identified by bright field imaging, the organoids included readily identifiable neural structures including cerebral cortex, cephalic flexure, and optic stalk (compare, Grey's Anatomy Textbook). The gene expression pattern in the neural organoid was >98% concordant with those of the adult human brain reference (Clontech, #636530). The organoids also expressed genes in a developmentally organized manner described previously (e.g. for the midbrain mesencephalic dopaminergic neurons; Blaese et al., Genetic control of midbrain dopaminergic neuron development. Rev Dev Biol. 4(2): 113-34, 2015). The structures also stained positive for multiple neural specific markers (dendrites, axons, nuclei), cortical neurons (Doublecortin), midbrain dopamine neurons (Tyrosine Hydroxylase), and astrocytes (GFAP) as shown by immunohistology).

All human neural organoids were derived from iPSCs of fibroblast origin (from the Coriell Biorepository, NJ). The development of a variety of brain structures was characterized in the organoids. Biomarkers specific for particular regions of human brain were detected as set forth in U.S. Pat. No. 11,435,890, incorporated by reference in its entirety herein.

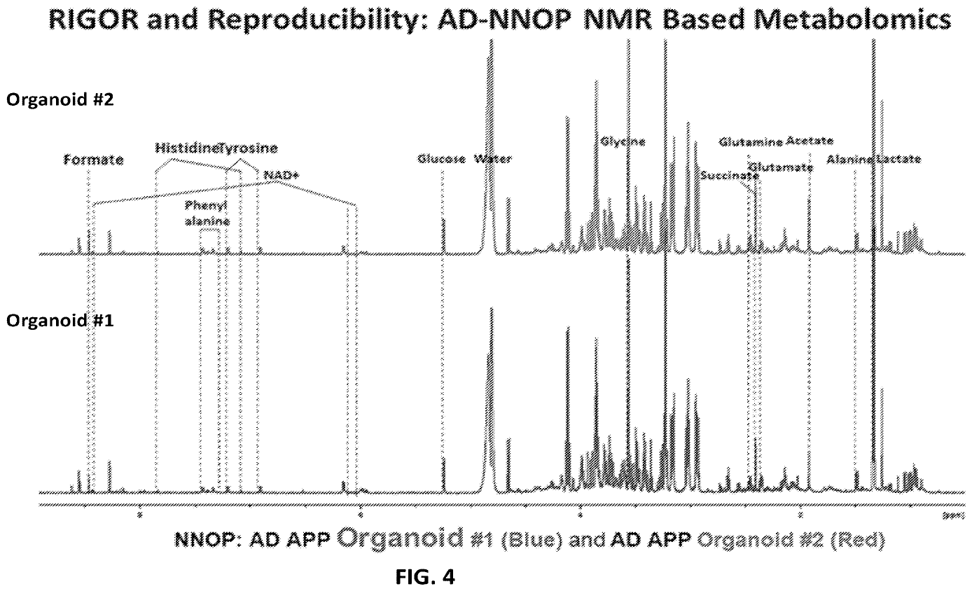

The results of transcriptomics experiments are shown in FIG. 4 and were performed as described herein and in U.S. Pat. No. 11,435,890, incorporated by reference in its entirety. Briefly, neural organoids were cultured in media after neural differentiation was initiated with addition of retinoic acid as per the published protocol (International patent application publication No. WO2017123791A1). Cultures were replenished with fresh media every week. Organoids were harvested after D0, D3, Week 1, Week 4 and Week 12 in culture.

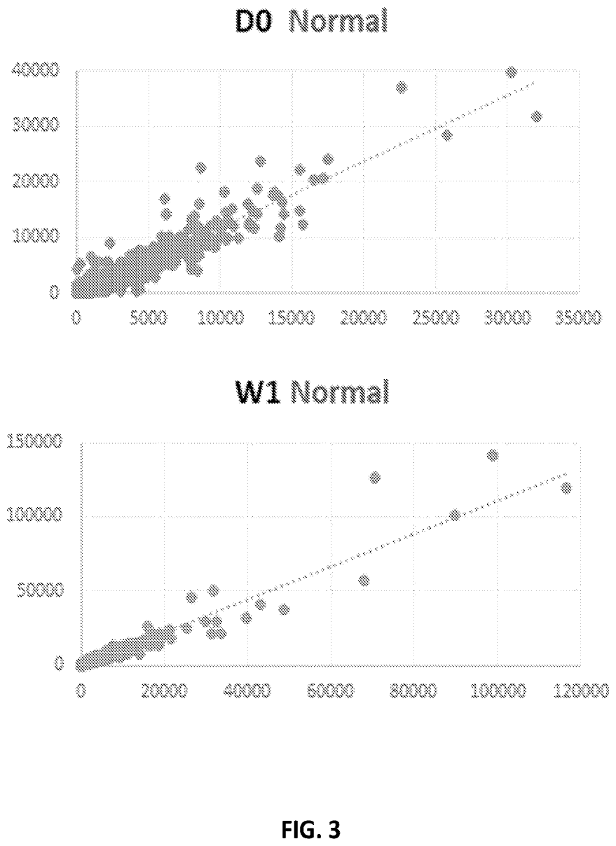

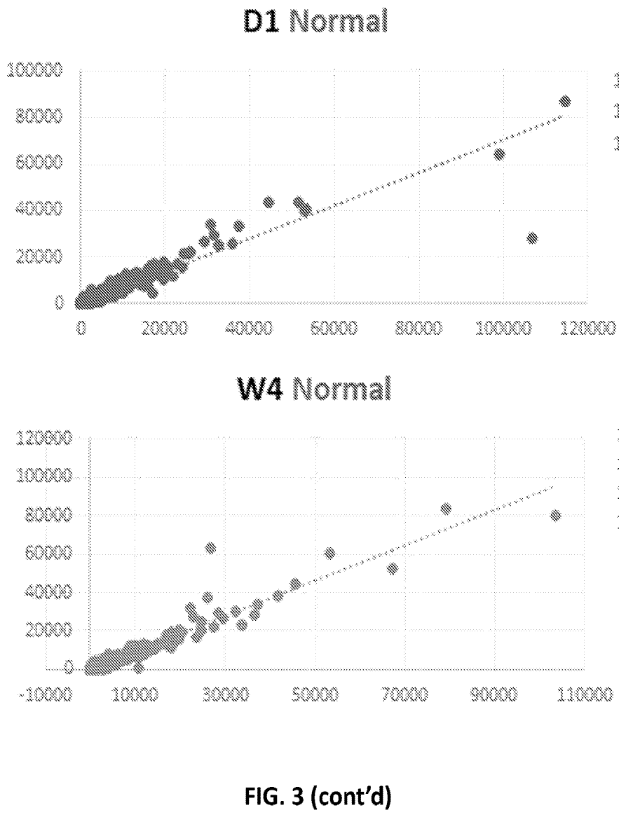

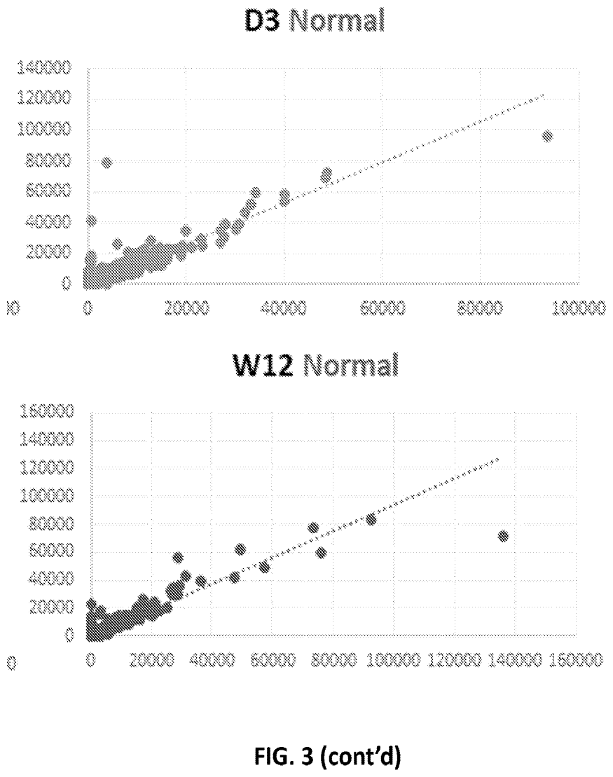

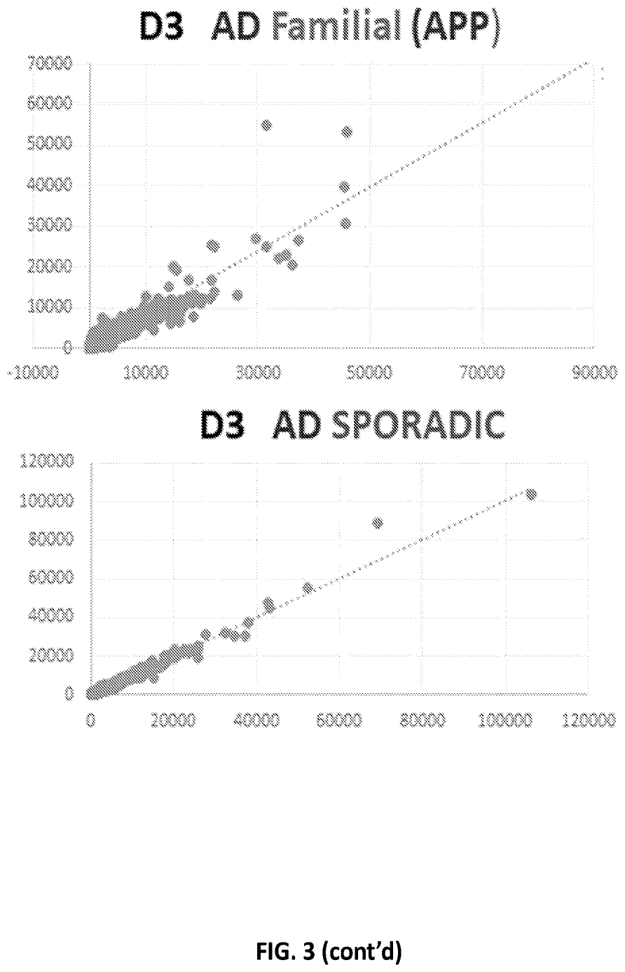

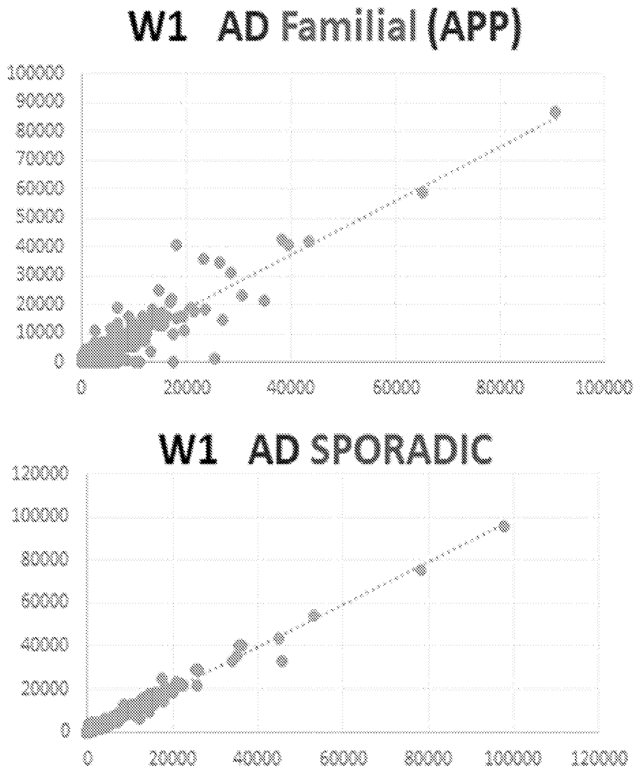

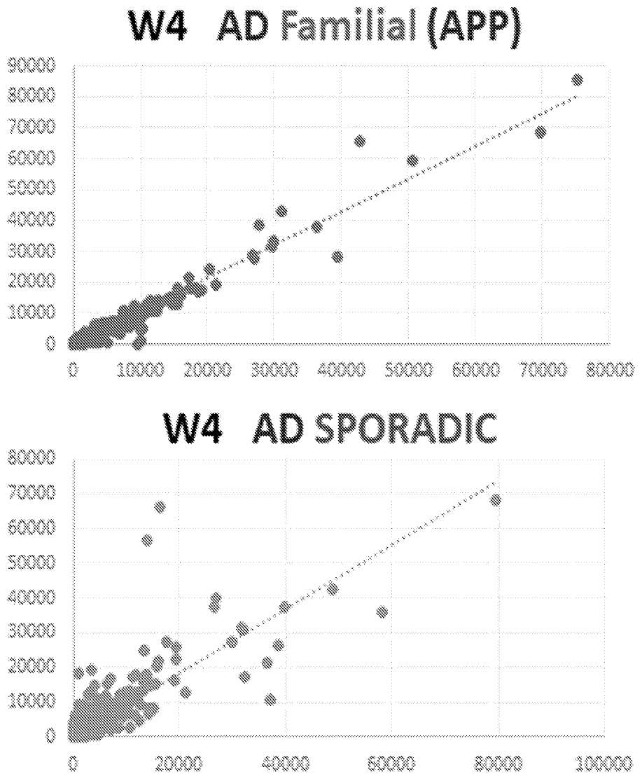

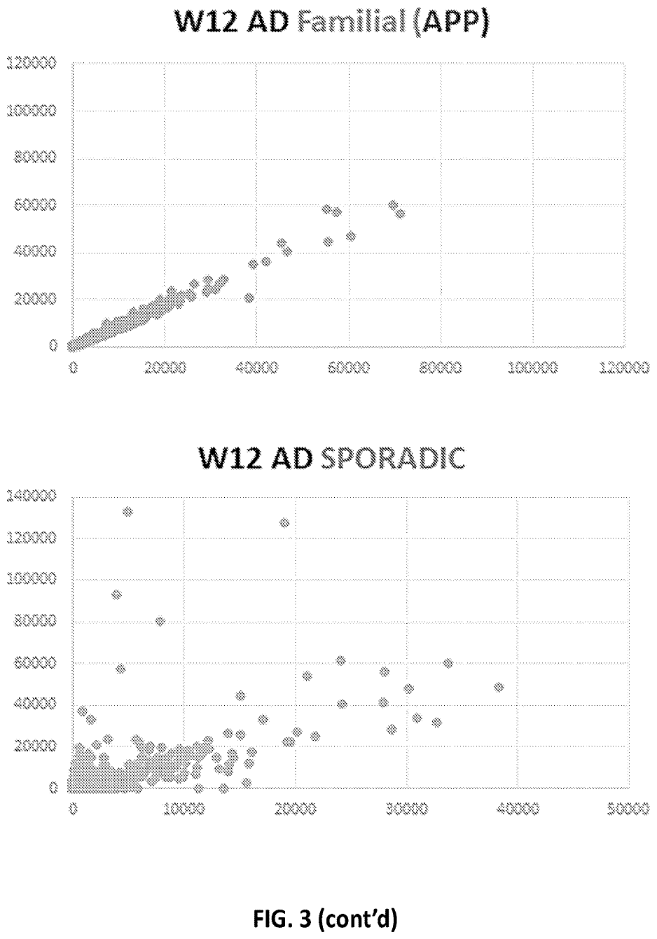

FIG. 3 showed transcriptomic data of about 15,000 genes of NNOP obtained from familial (APP) and sporadic (SPOR) patients in controlled clinical studies. Each dot represented RNA expression level of a gene measured in two randomly selected NNOP out of hundreds of NNOP. The variance was as low as <0.95 showing that there was little variability between independent organoid replicates, which demonstrated the reproducibility, replicability, and robustness of the NNOP model. Thus, NNOP models are a reliable model to study AD and comparative data analysis from NNOP platform between normal and dementia patient-derived samples permits identification of therapeutic targets and individualization of treatment decisions.

Indeed, thousands of differentially expressed genes (DEG) had been found in different AD-NNOP models compared to Normal-NNOP, and in AD-NNOP with drug treatment compared to control treatment. Out of these DEGs, some have been identified in Alzheimer's Patient Postmortem Brain to be associated with clinical symptoms and features. Note that the genes listed in Tables 1, 2A-2D, 3, 4, and 6A-6D are some representatives of clinically relevant DEG only but are not complete lists of all DEG found in the study.

In Table 2, expression of differentially expressed genes in four AD-NNOP neural organoids was compared to Normal-NNOP in culture for 12 weeks. RNA extraction and gene expression analysis by Ampliseq™ (Thermofisher). The four AD-NNOP models (AD-NNOP-APP; AD-NNOP-PSEN2; AD-NNOP-SPOR; and AD-NNOP-ApoE) were derived from AD patient donors and the iPSCs obtained from the Coreill Biorepository (NJ, USA). Log2 fold change with a positive value indicates a decrease in expression of an AD-NNOP gene when compared to Normal-NNOP and with a negative number an increase in gene expression. Thousands of genes in four AD-NNOP models were significantly altered when compared to Normal-NNOP including genes that were clinically relevant (Tables 2A-2D, not all significantly altered and clinically relevant genes were listed), confirming that the AD-NNOP models are reflective of the respective AD disease and can be used to study these diseases. Note that not the same genes were found significantly altered in each model, showing how these genes which correspond to clinical symptoms can vary across patients with different forms of AD. Additionally, in clinical settings, AD patients tend to have more than one symptom (comorbid symptoms) and some symptoms show up prior to the onset of cognitive failure. The data in Table 2 could assist in stratifying patients accordingly for personalized treatment and provide additional measurements to assess progress of AD prior to the onset of cognitive failure, as well as assess efficacy of candidate drugs.

Furthermore, the transcriptomic data collected from AD-NNOP model could also provide information regarding potential therapeutic targets. For examples, based on STRING network analysis, some DEGs in AD-NNOP-APP (compared to Normal-NNOP) were identified to interact with each other. Namely, changes in expression of five representative genes are shown in the following Table C. These genes were predicted to interact with each other; and thus, drugs that target receptors encoded by ADORA1 or ADORA2A would potentially affect ACTN4, CALM3, or SYNGR1.

TABLE C
Neural organoids were cultured in media after neural differentiation
was initiated with addition of retinoic acid as per the
published protocol (USPTO patent WO2017123791A1). The cultures
were replenished with fresh media every week. Organoids
were harvested after 12 weeks in culture and subject to
transcriptomic analysis. Results (log2 fold change) show
are normal-NNOP compared to AD-NNOP-APP.

| Gene_ID | base Mean | log2 Fold Change | p-value |
|---|---|---|---|
| ADORA1 | 150.83 | 1.94 | 0.0108 |
| ADORA2A | 30.95 | 2.17 | 0.0311 |
| ACTN4 | 1416.14 | −0.75 | 0.0303 |
| CALM3 | 9148.53 | 0.82 | 0.0010 |
| SYNGR1 | 404.15 | 1.85 | 0.0001 |

Additionally, based on the data collected from AD-NNOP models compared to Normal-NNOP and mathematical modeling, five key biomarkers identified in Table 4 were predicted to be effective therapeutic targets to treat AD. These were: CNR1 encoding endocannabinoid Anandamide Receptor; ADORA2A, ADORA1, ADORA2B all encoding adenosine receptors; and S1PR1 encoding sphingosine-1-phosphate-receptor. Thus, drugs that modulate these targets could correct the gene dysregulation in AD-NNOP models and clinical AD.

In conclusion, using in vitro NNOP to study AD disease was shown to be reproducible, robust, and accurately model the biomarkers found in variety of the disease forms which correspond to many clinical symptoms. The model would save time and cost compared to traditional animal models, and allow drug testing in diverse human donor patients, sex, and ancestry which is more aligned to FDA's standard of testing (Zushin et al., 2023, Journal of Clinical Investigation 133(21): e175824.

### Example 3: Testing Adenosine A2a Receptor Agonists for Efficacy in Human NNOP Model

Based on the results of Table 4, adenosine agonists (A_{2A}) were tested for effect on gene expression dysregulation in NNOP platform cells as set forth herein.

2-p-(2-Carboxyethyl) phenethylamino-5′-N-ethylcarboxamidoadenosine hydrochloride hydrate (CGS-21680) is a specific adenosine A_{2A} subtype receptor agonist (CAS Number: 124182-57-6; Molecular Weight: 535.98 (anhydrous basis), available from Sigma Chemical Co.). It is usually presented as an organic hydrochloride salt with a molecular weight of 536.0 g/M, is soluble up to 3.4 mg/mL in DMSO and 20 mg/mL in 45% (w/v) aq 2-hydroxypropyl-β-cyclodextrin.

In Table 1, the effect of A2A receptor agonist to reverse the aberrant expression of the biomarkers in AD disease was assessed. AD-NNOP-SPOR neural organoids for >12 weeks in culture were used to test the efficacy of the highly selective A2A Receptor agonist CGS-21680. CGS-21680 was tested at 1 μM; A2A receptor EC₅₀ of 110 nM). AD-NNOP were incubated with vehicle (0.1% DMSO; control) or CGS-21680 dissolved in DMSO at 1 μM for 4 days and harvested for RNA extraction and gene expression analysis by Ampliseq™ (Thermofisher). Log2 fold change with a positive value indicates a decrease in expression of an AD-NNOP gene when compared to Normal-NNOP and a negative number indicates an increase in gene expression. Changes in gene regulatory networks (GRN) as a consequence of activating ADORA2A receptor by CGS-21680 were investigated and the outcome was measured as differentially expressed genes (DEGs). 2023 genes were found to be differentially expressed in the CGS-21680 group compared to the control group in AD-NNOP-SPOR. As mentioned above, only a subset of these genes that were clinically relevant is shown in Table 1. The results show that CGS-21680 was able to invert the expression of the abnormal genes in AD and revert them back to normalcy (i.e. normalize expression of these genes). For example, expression of TBX3 gene was found to significantly increase in AD-NNOP-APP; AD-NNOP-PSEN2; AD-NNOP-SPOR; and AD-NNOP-ApoE models in Table 2A-2D compared to Normal-NNOP (indicated by negative log 2 fold change). However, addition of CGS-21680 decreased TBX3 expression compared to control treatment in AD-NNOP SPOR as indicated by a positive log 2 fold change (Table 1 and Table 5). Summary of the expression of TBX3 from Table 1 and Tables 2A-2D is provided in Table 5. Summary of the expression of the rest of the genes from Table 1 and Tables 2A-2D is provided in Table 6.

It is known that FAAH inhibitor PF-3845 will raise endogenous levels of endocannabinoid anandamide receptor agonist anandamide. Consequently, PF-3845 treatment can increase endocannabinoid anandamide receptor activity despite dementia-associated downregulation. To test the effect of FAAH inhibitor on AD, AD-NNOP-APP neural organoids for >12 weeks in culture were used to test the efficacy of the highly selective Fatty Acid Amide Hydroxylase (FAAH) inhibitor PF-3845 was tested at 10 UM). AD-NNOP-APP neural organoids were incubated with vehicle (0.1% DMSO; control) or PF-3845 dissolved in DMSO at 10 UM for 4 days and harvested for RNA extraction and gene expression analysis by Ampliseq™ (Thermofisher). Log2 fold change with a positive value indicates a decrease in expression of an AD-NNOP gene when compared to Normal-NNOP and with a negative number an increase in gene expression. Similar to Table 1, 2111 genes were found to be differentially expressed in the PF-3845 group compared to the control group in AD-NNOP-APP, but only a subset of these genes that was clinically relevant is shown in Table 3. The results in Table 3 showed that expression of the measured genes were decreased by PF-3845 compared to control treatment in AD-NNOP APP. The expression of these genes were known to be increased in AD-NNOP-APP compared to normal (data not shown). Thus, PF-3845 or FAAH inhibitor was effective in reverting the biomarkers of AD to normalcy.

### Example 4: Testing A2A Agonist and Combination with Other Drugs for Efficacy in Human NNOP Model

CGS-21680 is tested at a range of concentrations (dose range: 100 nM-1 microM; EC₅₀ of 110 nM) to identify the most effective dose at which a significant number of disease modifying genes are differentially expressed to ‘normalize’ their expression by epigenetic mechanism affecting gene regulatory networks in ADRD/MED. An adenosine A_{2A} receptor antagonist SCH-58261, a potent and selective antagonist for the adenosine A_{2A} receptor, with 50× selectivity for the adenosine A_{2A} receptor over other adenosine receptors (Zochi et al., 1996, J. Pharmacol. Exper. Ther. 276:398-404; dose range: 15-100 nM; IC₅₀ of 15 nM) by itself or in combination with CGS-21680 as specificity controls to antagonize the effects of the agonist CGS-21680. The effects of adenosine A_{2A} agonists can be enhanced by type IV phosphodiesterase inhibitors, such as Rolipram. Hence the effects of Rolipram (IC₅₀=800 nM; 1-10 microM) are tested by co-incubated with A_{2A} agonists. These experiments are performed at week 4 and 12 of development in culture. NNOPs are harvested after 1 week of exposure (at week 5, and at Week 13). Three independent experiments with replicates are performed and transcriptomic data analyzed by R-analysis.

Another adenosine A_{2A} receptor agonist, apadenoson (ATL-146e), chemical name methyl (1R,4r)-4-(3-(6-amino-9-((2R,3R,4S,5S)-5-(ethylcarbamoyl)-3,4-dihydroxytetrahydrofuran-2-yl)-9H-purin-2-yl) prop-2-yn-1-yl)cyclohexane-1-carboxylate; CAS Number: 250386-15-3 Molecular Weight: 486.529; Soluble in DMSO. MedKoo Biosciences, Inc., is a selective adenosine A_{2A} receptor agonist and a potent inhibitor of inflammation. Apadenoson is tested using a range of concentrations (dose range: 100 nM-1 microM; EC50 of 110 nM); to enhance the effects of adenosine apadenoson certain experiments are performed in the presence of Rolipram (1-10 microM; IC₅₀=800 nM). The experiments will be carried out and results statistically analyzed as described above. It is expected that agonists but not antagonists show positive results for ADRD/MED-related gene expression. Nevertheless, this prediction challenges existing studies in mouse model surrogates of AD. These expectations are contrary to certain published reports (Faivre et al., 2018, Front. Mol. Neurosci 11, doi.org/10.3389/fnmol.2018.00235) regarding the beneficial effects of a selective adenosine A_{2A} receptor antagonist in the APPswe/PS1dE9 mouse model of Alzheimer's disease. Use of adenosine A_{2A} receptor agonists is supported by results on expression levels of ADORA2A in two different ADRD/MED-NNOP models, to promote the ADORA2A anti-inflammatory signaling pathway and the differences in these expectations can be attributed to the differences between using human cell-derived NNOP analysis as opposed to transgenic mouse models known in the art. One way to test the significance of these differences and their relevance to treating ADRD/MED in humans is by using transcriptomics of the 5×FAD and APP/PS1 that have been published (De Bastiani et al., 2021, Transcriptomic similarities and differences between mouse models and human Alzheimer's disease, bioRxiv Jun. 25, 2021, doi.org/10.1101.06.09.447404). A comparison of the 5×FAD mouse published brain transcriptomic data to that of post-mortem brains from late onset AD (LOAD) patients (Annese et al., 2018, Scientific Reports 8:4282) shows little significant match (approximately 50 random genes possibly by chance) to the approximately 2124 genes that are dysregulated in human AD patients. In contrast, the ADRD/MED-APP-NNOP shows a match of 351 genes to the LOAD postmortem data and with a statistical p-value of 1e-103 of a match by chance alone. In addition, NNOP data is also well corroborated by other independent human clinical GWAS and postmortem studies (Blanchard et al., 2022, Nature 611:769-779; Baloni et al., 2022, Communications Biology 5:1074).

One of skill in the art will recognize that sequence data for the genes listed above can be obtained in publicly available gene databases such as GeneCards, GenBank, Malcard, Uniport and PathCard databases. The skilled worker will recognize these markers as set forth exemplarily herein to be human-specific marker proteins as identified, inter alia, in genetic information repositories such as GenBank; Accession Number. One having skill in the art will recognize that variants derive from the full length gene sequence.

TABLE 1
CGS-21680 alters expression of biomarkers in AD-NNOP SPOR model toward
normalcy. The biomarkers are found in Alzheimer's Patient Postmortem
Brain and associated with clinical symptoms and features.

| AD Patient | AD-NNOP | | | | | |
|---|---|---|---|---|---|---|
| Post Mortem | SPOR + | | log2 | | Clinical | |
| Brains | CGS- | Base | Fold | p- | Symptoms or | Reference |
| (Ref #1) | 21680 | Mean | Change | value | Features | # |
| ALOX15B | ALOX15B | 31.8 | −9.31 | 0.0112 | Inflammation | 2 |
| APLNR | APLNR | 357.9 | −5.96 | 0.0012 | CVID; Blood pressure, | 3 |
| | | | | | heart contractility | |
| | | | | | and heart failure | |
| ATOH7 | ATOH7 | 188.3 | −4.79 | 0.0047 | Vision and Retina | 4 |
| ATP6V1F | ATP6V1F | 60.1 | −3.56 | 0.0315 | Ceroid Deposition | 5 |
| BEST4 | BEST4 | 24.0 | −5.48 | 0.0059 | Vision | 6 |
| BHLHE41 | BHLHE41 | 231.6 | 1.65 | 0.0489 | Sleep Dysregulation | 7 |
| CHRM4 | CHRM4 | 311.9 | −3.74 | 0.0160 | Neuropsychiatric | 8 |
| | | | | | Behavior | |
| CSF1R | CSF1R | 79.2 | −4.26 | 0.0054 | Infectious | 9 |
| | | | | | Disease | |
| | | | | | Susceptibility | |
| DIO2 | DIO2 | 65.9 | 4.05 | 0.0097 | Thyroid Hormone | 10 |
| | | | | | Dysregulation | |
| DRD5 | DRD5 | 296.9 | −5.07 | 0.0041 | Attention | 11 |
| | | | | | Deficit- | |
| | | | | | Hyperactivity | |
| | | | | | Disorder | |
| GABRA1 | GABRA1 | 79.2 | −6.87 | 0.0048 | Memory and | 12 |
| | | | | | Cognitive | |
| | | | | | Impairment | |
| HTR1A | HTR 1A | 158.0 | −4.64 | 0.0157 | Generalized | 13 |
| | | | | | Anxiety | |
| KCNA5 | KCNA5 | 309.8 | −4.87 | 0.0102 | Insulin | 14 |
| | | | | | Resistance | |
| STC1 | STC1 | 1605.9 | 3.11 | 0.0011 | AD Cerebrospinal | 15 |
| | | | | | Fluid Biomarker | |
| STOM | STOM | 298.3 | 3.22 | 0.0081 | Anemia | 16 |
| TBX3 | TBX3 | 361.0 | 2.90 | 0.0056 | Cellular | 17 |
| | | | | | Senescence | |
| TCIRG1 | TCIRG1 | 54.0 | −5.13 | 0.0027 | Innate | 18 |
| | | | | | mmunity | |

TABLE 2A
APP Model. Differential gene expression of biomarkers in
AD-NNOP APP model compared to Normal-NNOP. All listed
biomarkers are found in Alzheimer's Patient Postmortem
Brain and associated with clinical symptoms and features.
AD-NNOP APP

| | | log2 | | |
|---|---|---|---|---|
| | base | Fold | p- | |
| | Mean | Change | value | Clinical Symptoms or Features |
| ALOX15B | | | | Inflammation |
| APLNR | | | | CVID; Blood pressure, heart |
| | | | | contractility and heart failure |
| ATOH7 | | | | Vision and Retina |
| ATP6V1F | | | | Ceroid Deposition |
| BEST4 | | | | Vision |
| BHLHE41 | 67.82 | −2.16 | 0.05 | Sleep Dysregulation |
| CHRM4 | 51.20 | 2.29 | 0.01 | Neuropsychiatric Behavior |
| CSF1R | | | | Infectious Disease Susceptibility |
| DIO2 | 79.17 | 2.71 | 0.01 | Thyroid Hormone Dysregulation |
| DRD5 | | | | Attention Deficit-Hyperactivity |
| | | | | Disorder |
| GABRA1 | | | | Memory and Cognitive |
| | | | | Impairment |
| HTR1A | | | | Generalized Anxiety |
| KCNA5 | | | | Insulin Resistance |
| STC1 | | | | AD Cerebrospinal Fluid |
| | | | | Biomarker |
| STOM | | | | Anemia |
| TBX3 | 84.50 | −3.97 | 0.01 | Cellular Senescence |
| TCIRG1 | | | | Innate Immunity |

TABLE 2B
PSEN2 Model. Differential gene expression of biomarkers in
AD-NNOP-PSEN2 model compared to Normal-NNOP. All listed
biomarkers are found in Alzheimer's Patient Postmortem
Brain and associated with clinical symptoms and features.
AD-NNOP PSEN2

| | | log2 | | |
|---|---|---|---|---|
| | base | Fold | p- | |
| | Mean | Change | value | Clinical Symptoms or Features |
| ALOX15B | | | | Inflammation |
| APLNR | 23.89 | 2.30 | 0.05 | CVID; Blood pressure, heart |
| | | | | contractility and heart failure |
| ATOH7 | 22.90 | 3.59 | 0.01 | Vision and Retina |
| ATP6V1F | | | | Ceroid Deposition |
| BEST4 | | | | Vision |
| BHLHE41 | 319.90 | −4.11 | 0.00 | Sleep Dysregulation |
| CHRM4 | | | | Neuropsychiatric Behavior |
| CSF1R | | | | Infectious Disease Susceptibility |
| DIO2 | 61.24 | 9.22 | 0.00 | Thyroid Hormone Dysregulation |
| DRD5 | 34.04 | 2.93 | 0.02 | Attention Deficit-Hyperactivity |
| | | | | Disorder |
| GABRA1 | | | | Memory and Cognitive |
| | | | | Impairment |
| HTR1A | 46.34 | 2.19 | 0.00 | Generalized Anxiety |
| KCNA5 | 25.86 | 3.33 | 0.02 | Insulin Resistance |
| STC1 | | | | AD Cerebrospinal Fluid |
| | | | | Biomarker |
| STOM | 715.33 | −2.63 | 0.00 | Anemia |
| TBX3 | 363.46 | −5.47 | 0.00 | Cellular Senescence |
| TCIRG1 | 86.28 | −2.09 | 0.00 | Innate Immunity |

TABLE 2C
Sporadic AD (SPOR) Model. Differential gene expression of biomarkers
in AD-NNOP-SPOR model compared to Normal-NNOP. All listed
biomarkers are found in Alzheimer'sPatient Postmortem Brain and
associated with clinical symptoms and features.
AD-NNOP SPOR

| | | log2 | | |
|---|---|---|---|---|
| | base | Fold | p- | |
| | Mean | Change | value | Clinical Symptoms or Features |
| ALOX15B | | | | Inflammation |
| APLNR | | | | CVID; Blood pressure, heart |
| | | | | contractility and heart failure |
| ATOH7 | | | | Vision and Retina |
| ATP6V1F | | | | Ceroid Deposition |
| BEST4 | | | | Vision |
| BHLHE41 | 73.36 | −2.05 | 0.03 | Sleep Dysregulation |
| CHRM4 | | | | Neuropsychiatric Behavior |
| CSF1R | 70.39 | −3.61 | 0.04 | Infectious Disease Susceptibility |
| DIO2 | | | | Thyroid Hormone Dysregulation |
| DRD5 | | | | Attention Deficit-Hyperactivity |
| | | | | Disorder |
| GABRA1 | | | | Memory and Cognitive |
| | | | | Impairment |
| HTR1A | | | | Generalized Anxiety |
| KCNA5 | | | | Insulin Resistance |
| STC1 | 825.92 | −2.95 | 0.00 | AD Cerebrospinal Fluid |
| | | | | Biomarker |
| STOM | 681.94 | −2.90 | 0.00 | Anemia |
| TBX3 | 147.50 | −4.45 | 0.00 | Cellular Senescence |
| TCIRG1 | | | | Innate Immunity |

TABLE 2D
ApoE Model. Differential gene expression of biomarkers in
AD-NNOP-ApoE model compared to Normal-NNOP. All
listed biomarkers are found in Alzheimer's Patient Postmortem
Brain and associated with clinical symptoms and features.
AD-NNOP ApoE

| | | log2 | | |
|---|---|---|---|---|
| | base | Fold | p | |
| | Mean | Change | value | Clinical Symptoms or Features |
| ALOX15B | | | | Inflammation |
| APLNR | 32.17 | 2.51 | 0.05 | CVID; Blood pressure, heart |
| | | | | contractility and heart failure |
| ATOH7 | | | | Vision and Retina |
| ATP6V1F | 25.65 | 3.06 | 0.01 | Ceroid Deposition |
| BEST4 | | | | Vision |
| BHLHE41 | | | | Sleep Dysregulation |
| CHRM4 | 135.44 | 2.52 | 0.00 | Neuropsychiatric Behavior |
| CSF1R | | | | Infectious Disease Susceptibility |
| DIO2 | | | | Thyroid Hormone Dysregulation |
| DRD5 | 36.44 | 3.98 | 0.01 | Attention Deficit-Hyperactivity |
| | | | | Disorder |
| GABRA1 | 11.89 | 5.79 | 0.02 | Memory and Cognitive |
| | | | | Impairment |
| HTR1A | 21.62 | 5.03 | 0.00 | Generalized Anxiety |
| KCNA5 | 86.74 | 2.14 | 0.00 | Insulin Resistance |
| STC1 | 374.91 | −2.01 | 0.00 | AD Cerebrospinal Fluid |
| | | | | Biomarker |
| STOM | 285.75 | −2.71 | 0.00 | Anemia |
| TBX3 | 223.59 | −8.03 | 0.00 | Cellular Senescence |
| TCIRG1 | | | | Innate Immunity |

TABLE 3
AD Model. PF-3845 alters expression of biomarkers
in AD-NNOP-APP model toward normalcy.

| AD Patient | AD- | | | | | |
|---|---|---|---|---|---|---|
| Post Mortem | NNOP | | log2 | | Clinical | |
| Brains | APP + | Base | Fold | p- | Symptoms or | Reference |
| (Ref #1) | PF 3845 | Mean | Change | value | Features | # |
| ADM | ADM | 55.07 | 2.14 | 0.02 | Hypertension | 2 |
| BGN | BGN | 14.24 | 2.50 | 0.05 | Impaired brain | 3 |
| | | | | | glucose | |
| | | | | | metabolism | |
| CD36 | CD36 | 53.04 | 2.86 | 0.00 | CVID; Heart | 4 |
| | | | | | Disease | |
| MAP3K19 | MAP3K19 | 19.52 | 2.56 | 0.03 | Episodic Memory | 5 |
| SLC16A3 | SLC16A3 | 56.17 | 2.05 | 0.02 | Impaired brain | 6 |
| | | | | | glucose | |
| | | | | | metabolism | |

TABLE 4
Key Therapeutic Biomarkers Dysregulated in AD-NNOP Models

| | | |
|---|---|---|
| base | log2 Fold | |
| Mean | Change | p-value |

Endocannabioid Anandamide Receptor
CNR1

| | | | |
|---|---|---|---|
| AD-APP | 638.69 | 2.77 | 7.547E−12 |
| AD-PSEN2 | 498.28 | 7.05 | 1.74E−36 |
| AD-SPOR | 431.50 | 3.93 | 0.0019011 |
| AD-ApoE | 619.34 | 2.20 | 2.176E−21 |

Adenosine Receptors
ADORA2A

| | | | |
|---|---|---|---|
| AD-APP | | | |
| AD-PSEN2 | 37.83 | 2.21 | 0.0270773 |
| AD-SPOR | 30.95 | 2.17 | 0.031057 |
| AD-ApoE | 619.34 | 2.20 | 2.176E−21 |

ADORA1

| | | | |
|---|---|---|---|
| AD-APP | 150.83 | 1.94 | 0.0107792 |
| AD-PSEN2 | 135.05 | 2.19 | 1.111E−05 |
| AD-SPOR | 104.32 | 2.77 | 0.0002154 |
| AD-ApoE | 141.79 | 2.06 | 3.47E−05 |

ADORA2B

| | | | |
|---|---|---|---|
| AD-APP | | | |
| AD-PSEN2 | 213.46 | −1.17 | 0.0423331 |
| AD-SPOR | 270.49 | −2.02 | 0.0423331 |
| AD-ApoE | | | |

Sphingosine-1-phosphate receptor
S1PR1

| | | | |
|---|---|---|---|
| AD-APP | | | |
| AD-PSEN2 | 396.32 | −3.12 | 1.012E−15 |
| AD-SPOR | 305.92 | −3.02 | 0.0060121 |
| AD-ApoE | | | |

TABLE 5
Underlined numbers indicate dysfunctional gene expression. Bold numbers indicate therapeutic
drug efficacy. Comparison of TBX3 gene expression from Table 1 and Tables 2A-2D. Addition
of CGS-21680 suppressed TBX3 (positive log2 fold change, bold) while expression of TBX3
was increased in the AD models (negative log2 fold change, underlined).

| | base | log2 Fold | | |
|---|---|---|---|---|
| | Mean | Change | p-value | Clinical Symptoms or Features |
| Dysfunctional | | | | |
| Gene Expression | | | | |
| AD-NNOP APP | | | | |
| TBX3 | 84.50 | −3.97 | 0.01 | Cellular Senescence |
| AD-NNOP PSEN2 | | | | |
| TBX3 | 363.46 | −5.47 | 0.00 | Cellular Senescence |
| AD-NNOP SPOR | | | | |
| TBX3 | 147.50 | −4.45 | 0.00 | Cellular Senescence |
| AD-NNOP ApoE | | | | |
| TBX3 | 223.59 | −8.03 | 2.46191E−16 | Cellular Senescence |
| Therapeutic Drug Efficacy | | | | |
| AD-NNOP SPOR + CGS-21680 | | | | |
| TBX3 | 361.0 | 2.90 | 0.0056 | Cellular Senescence |

TABLE 6A
SPOR. Underlined numbers indicate dysfunctional gene expression. Bold numbers indicate therapeutic
drug efficacy. Comparison of all gene expression from Table 1 and Tables 2A-2D. Addition
of CGS-21680 inverted the expression of the dysregulated genes in AD-NNOP-SPOR model.

| | AD-NNOP SPOR | AD-NNOP SPOR | |
|---|---|---|---|
| | Minus drug | Plus Drug | |
| Genes | log2 Fold Change | log2 Fold Change | Clinical Symptoms or Features |
| ALOX15B | | −9.31 | Inflammation |
| APLNR | | −5.96 | CVID; Blood pressure, heart contractility and heart |
| | | | failure |
| ATOH7 | | −4.79 | Vision and Retina |
| ATP6V1F | | −3.56 | Ceroid Deposition |
| BEST4 | | −5.48 | Vision |
| BHLHE41 | −2.05 | 1.65 | Sleep Dysregulation |
| CHRM4 | | −3.74 | Neuropsychiatric Behavior |
| CSF1R | −3.61 | −4.26 | Infectious Disease Susceptibility |
| DIO2 | | 4.05 | Thyroid Hormone Dysregulation |
| DRD5 | | −5.07 | Attention Deficit-Hyperactivity Disorder |
| GABRA1 | | −6.87 | Memory and Cognitive Impairment |
| HTR1A | | −4.64 | Generalized Anxiety |
| KCNA5 | | −4.87 | Insulin Resistance |
| STC1 | −2.95 | 3.11 | AD Cerebrospinal Fluid Biomarker |
| STOM | −2.90 | 3.22 | Anemia |
| TBX3 | −4.45 | 2.90 | Cellular Senescence |
| TCIRG1 | | −5.13 | Innate Immunity |

TABLE 6B
APP. Underlined numbers indicate dysfunctional gene expression. Bold numbers indicate therapeutic
drug efficacy. Comparison of all gene expression from Table 1 and Tables 2A-2D. Addition of CGS-
21680 in AD-NNOP-SPOR inverted the expression of the dysregulated gene in AD-NNOP-APP model.

| | AD-NNOP APP | AD-NNOP SPOR | |
|---|---|---|---|
| | Minus Drug | Plus Drug | |
| Genes | log2 Fold Change | log2 Fold Change | Clinical Symptoms or Features |
| ALOX15B | | −9.31 | Inflammation |
| APLNR | | −5.96 | CVID; Blood pressure, heart contractility and heart failure |
| ATOH7 | | −4.79 | Vision and Retina |
| ATP6V1F | | −3.56 | Ceroid Deposition |
| BEST4 | | −5.48 | Vision |
| BHLHE41 | −2.16 | 1.65 | Sleep Dysregulation |
| CHRM4 | 2.29 | −3.74 | Neuropsychiatric Behavior |
| CSF1R | | −4.26 | Infectious Disease Susceptibility |
| DIO2 | 2.71 | 4.05 | Thyroid Hormone Dysregulation |
| DRD5 | | −5.07 | Attention Deficit-Hyperactivity Disorder |
| GABRA1 | | −6.87 | Memory and Cognitive Impairment |
| HTR1A | | −4.64 | Generalized Anxiety |
| KCNA5 | | −4.87 | Insulin Resistance |
| STC1 | | 3.11 | AD Cerebrospinal Fluid Biomarker |
| STOM | | 3.22 | Anemia |
| TBX3 | −3.97 | 2.90 | Cellular Senescence |
| TCIRG1 | | −5.13 | Innate Immunity |

TABLE 6C
PSEN2. Underlined numbers indicate dysfunctional gene expression. Bold numbers
indicate therapeutic drug efficacy. Comparison of all gene expression from
Table 1 and Tables 2A-2D. Addition of CGS-21680 in AD-NNOP-SPOR inverted
the expression of the dysregulated gene in AD-NNOP-PSEN2 model.

| | AD-NNOP PSEN2 | AD-NNOP SPOR | |
|---|---|---|---|
| | Minus Drug | Plus Drug | |
| Genes | log2 Fold Change | log2 Fold Change | Clinical Symptoms or Features |
| ALOX15B | | −9.31 | Inflammation |
| APLNR | 2.30 | −5.96 | CVID; Blood pressure, heart contractility and heart |
| | | | failure |
| ATOH7 | 3.59 | −4.79 | Vision and Retina |
| ATP6V1F | | −3.56 | Ceroid Deposition |
| BEST4 | | −5.48 | Vision |
| BHLHE41 | −4.11 | 1.65 | Sleep Dysregulation |
| CHRM4 | | −3.74 | Neuropsychiatric Behavior |
| CSF1R | | −4.26 | Infectious Disease Susceptibility |
| DIO2 | 9.22 | 4.05 | Thyroid Hormone Dysregulation |
| DRD5 | 2.93 | −5.07 | Attention Deficit-Hyperactivity Disorder |
| GABRA1 | | −6.87 | Memory and Cognitive Impairment |
| HTR1A | 2.19 | −4.64 | Generalized Anxiety |
| KCNA5 | 3.33 | −4.87 | Insulin Resistance |
| STC1 | | 3.11 | AD Cerebrospinal Fluid Biomarker |
| STOM | −2.63 | 3.22 | Anemia |
| TBX3 | −5.47 | 2.90 | Cellular Senescence |
| TCIRG1 | −2.09 | −5.13 | Innate Immunity |

TABLE 6D
ApoE. Underlined numbers indicate dysfunctional gene expression. Bold numbers
indicate therapeutic drug efficacy. Comparison of all gene expression from
Table 1 and Tables 2A-2D. Addition of CGS-21680 in AD-NNOP-SPOR inverted
the expression of the dysregulated gene in AD-NNOP-ApoE model.

| | AD-NNOP ApoE | AD-NNOP SPOR | |
|---|---|---|---|
| | Minus drug | Plus Drug | |
| Genes | log2 Fold Change | log2 Fold Change | Clinical Symptoms or Features |
| ALOX15B | | −9.31 | Inflammation |
| APLNR | 2.51 | −5.96 | CVID; Blood pressure, heart contractility and heart |
| | | | failure |
| ATOH7 | | −4.79 | Vision and Retina |
| ATP6V1F | 3.06 | −3.56 | Ceroid Deposition |
| BEST4 | | −5.48 | Vision |
| BHLHE41 | | 1.65 | Sleep Dysregulation |
| CHRM4 | 2.52 | −3.74 | Neuropsychiatric Behavior |
| CSF1R | | −4.26 | Infectious Disease Susceptibility |
| DIO2 | | 4.05 | Thyroid Hormone Dysregulation |
| DRD5 | 3.98 | −5.07 | Attention Deficit-Hyperactivity Disorder |
| GABRA1 | 5.79 | −6.87 | Memory and Cognitive Impairment |
| HTR1A | 5.03 | −4.64 | Generalized Anxiety |
| KCNA5 | 2.14 | −4.87 | Insulin Resistance |
| STC1 | −2.01 | 3.11 | AD Cerebrospinal Fluid Biomarker |
| STOM | −2.71 | 3.22 | Anemia |
| TBX3 | −8.03 | 2.90 | Cellular Senescence |
| TCIRG1 | | −5.13 | Innate Immunity |

### OTHER EMBODIMENTS

From the foregoing description, it will be apparent that variations and modifications can be made to the invention described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

Having described the invention in detail and by reference to specific aspects and/or embodiments thereof, it will be apparent that modifications and variations are possible without departing from the scope of the invention defined in the appended claims. More specifically, although some aspects of the present invention may be identified herein as particularly advantageous, it is contemplated that the present invention is not limited to these particular aspects of the invention. Percentages disclosed herein can vary in amount by ±10, 20, or 30% from values disclosed and remain within the scope of the contemplated invention.

Table 1 References:

1. Annese, A., Manzari, C., Lionetti, C. et al. Whole transcriptome profiling of Late-Onset
Alzheimer's Disease patients provides insights into the molecular changes involved in the
disease. Sci Rep 8, 4282 (2018).
2. Rui Tian, Xiangsheng Zuo, Jonathan Jaoude, Fei Mao, Jennifer Colby, Imad Shureiqi.
ALOX15 as a suppressor of inflammation and cancer: Lost in the link. Prostaglandins & Other
Lipid Mediators. Volume 132, September 2017, Pages 77-83
3. Yoshikawa, Masahiro MD, Asaba, Kensuke MD, Nakayama, Tomohiro. The APLNR
gene polymorphism rs7119375 is associated with an increased risk of development of essential
hypertension in the Chinese population. A meta-analysis. Medicine 99(50): p e22418,
Dec. 11, 2020.
4. Polo, V., Rodrigo, M., Garcia-Martin, E. et al. Visual dysfunction and its correlation with
retinal changes in patients with Alzheimer's disease. Eye 31, 1034-1041 (2017).
5. Yasir H Qureshi # 1, Penelope Baez # 1, Christiane Reitz . Endosomal Trafficking in
Alzheimer's Disease, Parkinson's Disease, and Neuronal Ceroid Lipofuscinosis. Mol Cell Biol.
2020 Sep. 14; 40(19).
6. A Renata Pellegrino, et al., Novel BHLHE41 Variant is Associated with Short Sleep and
Resistance to Sleep Deprivation in Humans. Sleep. 2014 Aug. 1; 37(8): 1327-1336.
7. Geda Y E, Roberts R O, Knopman D S, Petersen R C, Christianson T J, Pankratz V S, et al.
Prevalence of neuropsychiatric symptoms in mild cognitive impairment and normal cognitive
aging: population-based study. Arch Gen Psychiatry. 2008; 65: 1193-1198.
8. Anthony L. Komaroff, Can Infections Cause Alzheimer Disease? JAMA.
2020; 324(3): 239-240. doi: 10.1001/jama.2020.4085.
9. T Zaldy S. Tan, MD, MPH; Alexa Beiser, PhD; Ramachandran S. Vasan, MD; et al.
Thyroid Function and the Risk of Alzheimer Disease. Arch Intern Med. 2008; 168(14): 1514-1520.
10. Leffa, D. T., Ferrari-Souza, J. P., Bellaver, B. et al. Genetic risk for attention-
deficit/hyperactivity disorder predicts cognitive decline and development of Alzheimer's disease
pathophysiology in cognitively unimpaired older adults. Mol Psychiatry (2022).
11. Yilan Xu, Manna Zhao, Yuying Han, and Heng Zhang. GABAergic Inhibitory Interneuron
Deficits in Alzheimer's Disease: Implications for Treatment. Front Neurosci. 2020; 14: 660.
12. Mario F Mendez. The Relationship Between Anxiety and Alzheimer's Disease. J
Alzheimers Dis Rep. 2021 Mar. 8; 5(1): 171-177.
13. Derek Kellar, Suzanne Craft. Brain insulin resistance in Alzheimer's disease and related
disorders: mechanisms and therapeutic approaches. The LANCET; Neurology. VOLUME 19,
ISSUE 9, P758-766, SEPTEMBER 2020.
14. Pashtun Shahim, Kaj Blennow, Per Johansson, Johan Svensson, Simone Lista, Harald
Hampel, Leif Christer Andersson, Henrik Zetterberg. Neuromolecular Med. 2017 March; 19(1): 154-
160.
15. Pu Wang 1, Xiao-Long Li 1, Zhi-Hua Cao. STC1 ameliorates cognitive impairment and
neuroinflammation of Alzheimer's disease mice via inhibition of ERK1/2 pathway.
Immunobiology. Volume 226, Issue 3, May 2021, 152092.
16. Hemoglobin and anemia in relation to dementia risk and accompanying changes on
brain MRI. Frank J. Wolters, Hazel I. Zonneveld, Silvan Licher, Lotte G. M. Cremers. Neurology.
Aug. 27, 2019; 93 (9).
17. Rui-Ming Liu. Aging, Cellular Senescence, and Alzheimer's Disease. Int J Mol Sci. 2022
February; 23(4): 1989.
18. Song, Q., Meng, B., Xu, H. et al. The emerging roles of vacuolar-type ATPase-
dependent Lysosomal acidification in neurodegenerative diseases. Transl Neurodegener 9, 17
(2020).

Table 3 references:

1. Annese, A., Manzari, C., Lionetti, C. et al. Whole transcriptome profiling of Late-Onset
Alzheimer's Disease patients provides insights into the molecular changes involved in the
disease. Sci Rep 8, 4282 (2018).
2. Yoshikawa, Masahiro MD, Asaba, Kensuke MD, Nakayama, Tomohiro. The APLNR
gene polymorphism rs7119375 is associated with an increased risk of development of essential
hypertension in the Chinese population. A meta-analysis. Medicine 99(50): p e22418,
Dec. 11, 2020.
3. Tyler C. Hammond and Ai-Ling Lin. Glucose Metabolism is a Better Marker for
Predicting Clinical Alzheimer's Disease than Amyloid or Tau. J Cell Immunol. 2022; 4(1): 15-18.
4. Joshua M. Tublin, Jeremy M. Adelstein, Federica del Monte, Colin K. Combs and Loren
E. Wold. Getting to the Heart of Alzheimer Disease. Circulation Research. 2019; 124: 142-149.
5. Matthew J. Huentelman, Ignazio S. Piras, Ashley L. Siniard, Matthew D. De Both, Ryan
F. Richholt, Chris D. Balak, 1 Pouya Jamshidi, Eileen H. Bigio, Sandra Weintraub, Emmaleigh T.
Loyer. Associations of MAP2K3 Gene Variants With Superior Memory in SuperAgers. Front
Aging Neurosci. 2018; 10: 155.
6. Saikat Dewanjee. Altered glucose metabolism in Alzheimer's disease: Role of
mitochondrial dysfunction and oxidative stress. Free Radical Biology and Medicine. Volume
193, Part 1, 20 Nov. 2022, Pages 134-157

What is claimed is:

1. A method for reducing or ameliorating disease severity in an individual having dementia comprising administering to the individual a therapeutically effective amount of a pharmaceutical composition comprising a drug capable of reducing or correcting dysfunctional expression of genes related to dementia in vitro in a neural organoid platform and thereby reducing or ameliorating disease severity in an individual having dementia.

2. The method of claim 1, wherein the individual has dementia further characterized as Alzheimer's disease, Alzheimer's Disease Related Dementia (ADRD), Mixed Etiology Dementia (MED), Lewy Body dementia, fronto-temporal dementia, or vascular contributors to dementia.

3. The method of claim 1, wherein the genes related to ADRD or MED for which the drug is capable of reducing or correcting dysfunctional expression are set forth in Tables 1, 3, C, and 4.

4. The method of claim 1, 2, or 3, wherein the drug is an agonist of an adenosine A2a receptor. (Genbank Accession No. NP_001265429.1)

5. The method of claim 4, wherein the adenosine A2a receptor is encoded by an ADORA2A gene (Genbank Accession No. NM_001278497.2).

6. The method of claim 5, wherein the drug is methyl (1R,4r)-4-(3-(6-amino-9-((2R,3R,4S,5S)-5-(ethylcarbamoyl)-3,4-dihydroxytetrahydrofuran-2-yl)-9H-purin-2-yl) prop-2-yn-1-yl)cyclohexane-1-carboxylate (apadenoson).

7. The method of claim 5, wherein the drug is 2-p-(2-Carboxyethyl) phenethylamino-5′-N-ethylcarboxamidoadenosine hydrochloride hydrate (CGS-21680).

8. A pharmaceutical composition of a drug capable of reducing or ameliorating disease severity in an individual having dementia comprising a therapeutically effective amount of the pharmaceutical composition comprising a drug capable of reducing or correcting dysfunctional expression of genes related to dementia in vitro in a neural organoid platform that thereby reduces or ameliorates disease severity in an individual having dementia.

9. The pharmaceutical composition of claim 8, wherein the individual has dementia further characterized as Alzheimer's disease, Alzheimer's Disease Related Dementia (ADRD), Mixed Etiology Dementia (MED), Lewy Body dementia, fronto-temporal dementia, or vascular contributors to dementia.

10. The pharmaceutical composition of claim 8 or 9, wherein the genes related to ADRD or MED for which the drug is capable of reducing or correcting dysfunctional expression are set forth in Tables 1, 3, C, and 4.

11. The pharmaceutical composition of claim 10, wherein the drug is an agonist of an adenosine A2a receptor. (Genbank Accession No. NP_001265429.1)

12. The pharmaceutical composition of claim 10, wherein the adenosine A2a receptor is encoded by an ADORA2A gene (Genbank Accession No. NM_001278497.2).

13. The pharmaceutical composition of claim 10, wherein the drug is methyl (1R,4r)-4-(3-(6-amino-9-((2R,3R,4S,5S)-5-(ethylcarbamoyl)-3,4-dihydroxytetrahydrofuran-2-yl)-9H-purin-2-yl) prop-2-yn-1-yl)cyclohexane-1-carboxylate (apadenoson).

14. The pharmaceutical composition of claim 10, wherein the drug is 2-p-(2-Carboxyethyl) phenethylamino-5′-N-ethylcarboxamidoadenosine hydrochloride hydrate (CGS-21680).

15. A method for reducing or ameliorating disease severity in an individual having dementia comprising administering to the individual a therapeutically effective amount of a pharmaceutical composition comprising a drug capable of correcting dysfunctional expression of genes related to dementia in vitro in a neural organoid platform in combination with an FAAH inhibitor and thereby reducing or ameliorating disease severity in an individual having dementia.

16. The method of claim 15, wherein the individual has dementia further characterized as Alzheimer's disease, Alzheimer's Disease Related Dementia (ADRD), Mixed Etiology Dementia (MED), Lewy Body dementia, fronto-temporal dementia, or vascular contributors to dementia.

17. The method of claim 15 or 16, wherein the FAAH inhibitor is PF-3845.

18. The method of claim 15, 16, or 17, wherein the genes related to ADRD or MED for which the drug is capable of reducing or correcting dysfunctional expression are set forth in Table 3.

19. A pharmaceutical composition for use in the method of claim 15 or 16, comprising a therapeutically effective amount of a drug capable of reducing or correcting dysfunctional expression of genes related to dementia in vitro in a neural organoid platform in combination with an FAAH inhibitor that thereby reduces or ameliorates disease severity in an individual having ADRD or Mixed MED.

20. The pharmaceutical composition of claim 19 wherein the FAAH inhibitor is PF-3845, SSR411298, or PF-622.

21. A method for reducing or ameliorating disease severity in an individual having dementia comprising administering to the individual a therapeutically effective amount of a pharmaceutical composition comprising a drug capable of correcting dysfunctional expression of genes related to dementia in vitro in a neural organoid platform in combination with sphingosine-1-phosphate receptor 1 modulators and thereby reducing or ameliorating disease severity in an individual having dementia.

22. The method of claim 21, wherein the individual has dementia further characterized as Alzheimer's disease, Alzheimer's Disease Related Dementia (ADRD), Mixed Etiology Dementia (MED), Lewy Body dementia, fronto-temporal dementia, or vascular contributors to dementia.

23. The method of claim 21 or 22, wherein the sphingosine-1-phosphate receptor 1 modulators is fingolimod, ponesimed (ACT-12800), siponimed (Mayzant), ceralifimod (ONO-4641), or Amiselmod (MT-1303).

24. The method of claim 21, 22, or 23, wherein the genes related to ADRD or MED for which the drug is capable of reducing or correcting dysfunctional expression are set forth in Tables 1, 3, C, and 4.

25. A pharmaceutical composition for use in the method of claim 21 or 22, comprising a therapeutically effective amount of a drug capable of reducing or correcting dysfunctional expression of genes related to dementia in vitro in a neural organoid platform in combination with an FAAH inhibitor that thereby reduces or ameliorates disease severity in an individual having dementia.

26. The pharmaceutical composition of claim 25 wherein the sphingosine-1-phosphate receptor 1 modulators is fingolimod, siponimed (Mayzant), ponesimed (ACT 12800), ceralifimod (ONO-4641), or Amiselmod (MT-1303).

27. The method of claim 1, 2, or 3, wherein the drug is an antagonist of an adenosine A2b receptor. (GenBank Accession No. NP_00667.4)

28. The method of claim 27, wherein the adenosine A2b receptor is encoded by an ADORA2B gene (GenBank Accession No. NM00676.4).

29. The method of claim 27, wherein the genes related to ADRD or MED for which the drug is capable of reducing or correcting dysfunctional expression are set forth in Table 1, 3, C, and 4.

30. The method of claim 27, wherein the drug is PBF-1129 or MRS-1706.

31. A pharmaceutical composition of a drug capable of reducing or ameliorating disease severity in an individual having dementia comprising a therapeutically effective amount of the pharmaceutical composition comprising a drug capable of reducing or correcting dysfunctional expression of genes related to dementia in vitro in a neural organoid platform that thereby reduces or ameliorates disease severity in an individual having dementia wherein the drug is an antagonist of adenosine A2b receptor.

32. The pharmaceutical composition of claim 31, wherein the individual has dementia further characterized as Alzheimer's disease, Alzheimer's Disease Related Dementia (ADRD), Mixed Etiology Dementia (MED), Lewy Body dementia, fronto-temporal dementia, or vascular contributors to dementia.

33. The pharmaceutical composition of claim 31 or 32, wherein the genes related to ADRD or MED for which the drug is capable of reducing or correcting dysfunctional expression are set forth in Table 1, 3, C, and 4.
